(19) United States
(12) Patent Application Publication
(10) Pub. No.: US 2013/0209361 A1
(43) Pub. Date: Aug. 15, 2013

(54) PROCESS FOR PRODUCING RADIOHALOGENATED BIOCONJUGATES AND PRODUCTS THEREOF

(71) Applicants: Erik Arstad, London (GB); Ran Yan, London (GB)

(72) Inventors: Erik Arstad, London (GB); Ran Yan, London (GB)

(73) Assignee: UCL BUSINESS PLC, London (GB)

(21) Appl. No.: 13/817,785

(22) PCT Filed: Aug. 12, 2011
(86) PCT No.: PCT/GB2011/001216

§ 371 Date: May 3, 2013

(30) Foreign Application Priority Data
Aug. 20, 2010 (GB) 1014023.4

(51) Int. Cl. A61K 51/04

(52) U.S. Cl. CPC A61K 51/0453
USPC 424/1.85; 548/255; 570/216; 570/235; 435/7.1

(57) ABSTRACT

The present invention relates to a new synthetic process in which an alkyne and an azide react to form a radioisotopic bioconjugate construct. The reaction is particularly useful for producing compounds for use in imaging and radiotherapy applications. The present invention also provides bioconjugate labels and further relates to the use of these compounds in diagnostic and therapeutic methods. In addition, the invention provides a related process for introducing a radioisotopic halogen atom into a terminal alkyne.

### INTRODUCTION

Molecular imaging is an increasingly important tool for biomedical research and to guide treatment of disease. However, the applicability of such methods to studies of biological processes in vivo remains limited because no single imaging modality meets the need for high resolution, high sensitivity and deep tissue penetration.

Development of tracers with dual reporter groups would overcome some of the current restraints by forging synergies between complementary imaging techniques. For example, dual optical and nuclear tracers would allow imaging from single cells to the whole body, enabling detailed understanding of the behaviour of diagnostic tracers and therapeutic drugs in vivo, with the added benefit of enabling low cost, high through-put fluorescence assays for compound screening.

Dual optical and nuclear tracers would find further benefits in treatment of disease, where the nuclear reporter would enable whole body imaging of pathological processes, such as cancer, whereas the optical reporter would guide surgery/biopsies. Nuclear reporters in combination with MRI would allow quantification of contrast agents, which is unattainable with MRI alone. This could lead to development of improved contrast agents retaining the nuclear reporter in its non-radioactive form for routine applications. Combining optical reporters with CT and MRI probes would enable validation of the distribution at sub-cellular level, thereby providing a means to improve efficacy whilst reducing toxic effects.

It would also be desirable to combine reporter moieties with other moieties that have complementary functional properties for the intended application of the tracer. The combination of a reporter moiety with a moiety having binding specificity to a biomolecule of interest is well known, but a wealth of possibilities exists in relation to trifunctional constructs having two reporters together with such a binder moiety, or having a reporter group and two moieties that confer biological activity, or having a reporter group, a binder moiety and a third functional moiety designed further to modify the biochemical properties of the construct. For example, combining two complementary reporter moieties with a moiety having the ability to target a specific biomolecule allows control over precisely where the construct is to be directed. Combinations can also be envisaged of a reporter molecule with a targeting moiety and a moiety designed, for example, to modify the bioavailability or physicochemical properties of the construct (such as by PEGylation or glycolation), to assist in purification of the construct or to enable further chemical reactions to be effected with the construct.

The concept of obtaining a bioconjugate construct carrying more than two functional moieties is not new. For example, trifunctionalised peptide-based constructs are disclosed in WO 2004/064972. However, there remains a need for new trifunctionalised bioconjugate constructs, particularly for use in imaging and radiotherapeutic applications. Such constructs should be easy to synthesise, ideally enabling them to be made on site at, for example, a radiopharmacy, and metabolically stable, allowing them to be utilised reliably in the desired diagnostic and/or therapeutic application.

1,2,3-triazole adducts are an interesting class of materials owing to their excellent metabolic stability compared, for example, to the peptide-based constructs discussed above. Bioconjugates based on a triazole scaffold are known and include tracer substances comprising a single reporter group (for example, a radiolabelled or fluorescent probe) and a biologically active compound. The popularity of such bifunctional triazole-based bioconjugates is linked to their efficient synthesis from an azide (carrying a first functional moiety) and a terminal alkyne (carrying a second functional moiety). These starting materials are inert under most conditions, but cyclise readily to form triazoles in the presence of a copper (I) catalyst such as CuI. Reddy et al. (SynLett 2006 6 0957-0959) have also proposed that certain copper (II) catalysts may be capable of effecting this bimolecular reaction between the azide and the alkyne.

However, the possibility of creating trifunctional triazole constructs for use in imaging or therapeutic methods has not previously been proposed. The chemical methods currently available for making bifunctional triazoles cannot be used to construct and purify multi-functionalized tracers. The conventional two-component reaction to produce triazole bioconjugates from an azide and a terminal alkyne links together only two distinct functional moieties. Recently, Li et al (J Org Chem, 2008 83 3630-3633) and Wu et al (Synthesis 2005 1314) have both proposed three-component coupling reactions between an azide, an alkyne and a source of iodine, generating a triazole product that bears the two moieties bound to the azide and the alkyne, respectively, and also an iodine substituent. These reactions are potentially of interest in the imaging field since radioisotopic iodine (and astatine) can be used as a radiolabel in medical imaging methods.

In the method of Li et al, the reaction is carried out under anhydrous conditions using an excess of CuI together with an oxidising agent such as N-iodosuccinimide or N-bromosuccinimide. The method of Wu et al uses ICI as the iodination reagent and triethylamine as a ligand and base and is catalysed by CuI. However, in these reactions there is strong competition from the bimolecular reaction between the azide and alkyne only and it has not been possible to achieve specificity for the trimolecular (azide-alkyne-iodide) reaction.

It would not therefore be possible to use these procedures to create trifunctionalised triazoles that carry a radioisotopic halogen atom such as a radioisotopic iodine label together with two other functional moieties. There are numerous reasons for this, including that the reactions require non-aqueous conditions and at least stoichiometric quantities of iodine, whereas radioisotopic iodine sources are typically aqueous and comprise the radioisotope in only minute concentrations (of the order of picomolar). Under such conditions the undesirable bimolecular azide-alkyne reaction would be expected to be overwhelmingly dominant. Similarly, the lack of selectivity of the prior art techniques prevents formation of trifunctionalised triazoles having complex structures, such as bioconjugate constructs, because achieving purification from the bimolecular reaction by-products would be excessively demanding.

A need therefore exists for a process for producing stable multi-functionalised bioconjugate constructs suitable for use particularly in diagnostic and therapeutic imaging procedures. Such a process should be simple, reliable and capable of generating the product with high specificity, for example in a single three-component reaction. It would also be desirable to provide a class of multi-functionalised bioconjugate constructs that are metabolically stable and are readily obtainable using the production processes described.

### SUMMARY OF THE INVENTION

The present invention provides the following related embodiments [1] to [8]:

[1] A process for producing a triazole derivative represented by the formula (I)

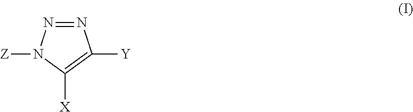

wherein
X represents a radioisotopic halogen atom; and
Y and Z each represent, independently of one another, a moiety which is
   a) a label, a chelating agent, a biologically active moiety or a moiety enabling purification of the derivative; or
   b) a reactive linker for linking the triazole derivative represented by the formula (I) to a label, a chelating agent, a biologically active moiety or a moiety enabling purification of the derivative;
      which process comprises (B) reacting an alkyne represented by the formula (II′)

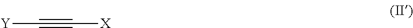

with an azide represented by the formula (III)

Z—N₃ (III)

said process being carried out in the presence of Cu(II) ions and a base.
[2] Use of Cu(II) ions for catalysing the reaction between (a) an alkyne represented by the formula (II)

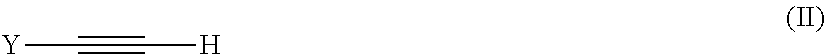

wherein Y is as defined in claim 1, (b) an azide represented by the formula (III) as defined in [1] and (c) a radioisotopic halogen anion, to produce a trisubstituted triazole derivative.
[3] A triazole derivative represented by the formula (I) as defined in [1], and wherein Y and Z each represent, independently of one another, a moiety which is a label, a chelating agent, a biologically active moiety or a moiety enabling purification of the derivative.
[4] A triazole derivative as defined in [3] for use in a diagnostic method practised on the human or animal body.
[5] A method of imaging carried out on a human or animal subject, which method comprises measuring the distribution in vivo of a triazole derivative as defined in [3].
[6] An in vitro assay method which comprises measuring the distribution in a biological sample of a triazole derivative as defined in [3].
[7] A triazole derivative as defined in [3] for use in a method of treatment of the human or animal body, wherein said triazole derivative comprises at least one drug moiety.
[8] A process for producing an alkyne represented by the formula (II′) as defined in [1], which process comprises reacting a terminal alkyne represented by the formula (II) as defined in [2] with a radioisotopic halogen anion, said process being carried out in the presence of Cu(II) ions and a base.

### FIGURES

FIG. 1 shows UV (negative trace) and Radio-HPLC (positive trace) chromatograms of a reaction mixture containing the compound [¹²⁵I]16 after 90 min reaction time as obtained according to the protocol described in Example 3.

FIG. 2 shows an image obtained in a fluorescent staining study of human colorecteral tumour tissue using monoclonal anti-CEA antibody coupled to the triazole reagent [125I]16 according to the protocol described in Example 3 with two Fluorophore/20 μL/mL of antibody A5B7.

FIG. 3 shows biodistribution of dual labelled antibodies (A5B7 and MOPC) in tumour bearing mice (specifically biodistribution of [¹²⁵I]3-A5B7 and [¹²⁵I]3-MOPC 6 h, 24 h and 48 h after injection). Values are expressed as a percentage of injected dose per gram of tissue (% ID/g)±SEM. Each time point represents the average uptake in 3 animals. For each body part, the bars are, from left to right, MOPC-6 hrs, MOPC-24 hrs, MOPC-48 hrs, A5B7-6 hrs, A5B7-24 hrs and A5B7-48 hrs

FIG. 4 demonstrates multiscale ex vivo imaging of antibody distribution in tumour section 6 h after i.v. injection of dual labelled antibody A5B7. A) Radioluminograph (bar showing counts per pixel from 0 (dark) to 6000 (white)); B) fluorescence image of adjacent tumor sections demonstrating uptake of dual labeled A5B7 6 hours post injection of 50 μg antibody; C) high power image demonstrating: distribution of dual labeled A5B7 antibody, Hoechst staining showing perfused blood vessels and CD31 staining showing blood vessel distribution; D) H&E showing tumor histology: viable tumor (V); necrosis (N). Numbered arrows indicate that the distribution of radioactivity (FIG. 4A) colocalized with the fluorescence signal (FIG. 4B), and demonstrate antibody uptake in viable tumor. Note that a full colour version of this Figure is publicly available in Angew. Chem. Int. Ed. 2011 50 6793-6795.

### DETAILED DESCRIPTION OF THE INVENTION

The present invention is concerned with the production of multi-functionalised triazole derivatives for use in imaging, chemical sensors and/or therapeutic applications. Constructs consisting of at least two discrete moieties that each have biological relevance are often referred to in the art as bioconjugates. The term bioconjugate has a broad meaning: typically the construct contains two or more discrete biomolecules, but the term is also applied to constructs containing a single biomolecule and at least one other moiety of biological significance, which could for example be a label to assist in tracing the construct during use or a substance that modifies the chemical or physical properties of the molecule in a way that has some biological significance, for example by altering the bioavailability of the construct. As used herein, the term biomolecule refers to any organic molecule that is produced by a living organism or any substance which substantially mimics the relevant properties of such a molecule. The triazole derivative represented by the formula (I) is preferably a bioconjugate.

Reference is made to the related chemical reactions and bioconjugate applications described in the following academic articles: “Click chemistry under non-classical reaction conditions” Chem. Soc. Rev. 2010 39 1280-1920; “Click labelling in PET radiochemistry” J. Label Compd. Radiopharm. 2009 52 407-414; “Synthesis and Applications of Biomedical and Pharmaceutical Polymers via Click Chemistry Methodologies” Online review article from Bioconjugate Chem. DOI 10.1021/bc900087a; “Fluorescent chemosensors for Zn²⁺” Chem. Soc. Rev. 2010 39 1996-2006; “Clickable Gold Nanoparticles as the Building Blocks of Nanobioprobes” Langmuir 2010 26(12) 10171-10176; “Hybrid PET-optical imaging using targeted probes” PNAS 107(17) 2010 7910-7915; “Clickable affinity ligands for effective separation of glycoproteins” Journal of Chromatography A, 1217 (2010) 3635-3641; “Copper(I) catalyzed cycloaddition of organic azides and 1-iodoalkynes” Angew. Chem. Int. Ed. 2009 48 8018-8021; “Acceleration of Cu(I)-mediated Huisgen 1,3-dipolar cycloaddition by histidine derivatives” Tetrahedron Letters 48 2007 6475-6479; “Metal- and base-free three-component reaction of ynones, sodium azide and alkyl halides: highly regioselective synthesis of 2,4,5-trisubstituted 1,2,3-NH-triazoles” Synlett 2010 11 1617-1622; “Organic/inorganic nanoobjects with controlled shapes from gelable triblock copolymers” Polymer 51 2010 2809-2817; and “A facile strategy for the fabrication of highly stable superhydrophobic cotton fabric using amphiphilic fluorinated triblock azide copolymers” Polymer 51 2010 1940-1946. These articles illustrate the breadth over which the principles of the present invention can be applied and are each herein incorporated by reference in their entirety.

As used herein, a triazole means a 1,2,3-triazole.

As used herein, the term “alkyl” includes both saturated straight chain and branched alkyl groups. Preferably, an alkyl group is a C₁₋₂₀ alkyl group, more preferably a C₁₋₁₅, more preferably still a C₁₋₁₂ alkyl group, more preferably still, a C₁₋₆alkyl group, and most preferably a C₁₋₄ alkyl group. Particularly preferred alkyl groups include, for example, methyl, ethyl, propyl, isopropyl, butyl, isobutyl, tert-butyl, pentyl and hexyl. The term “alkylene” should be construed accordingly.

As used herein, the term “alkenyl” refers to a group containing one or more carbon-carbon double bonds, which may be branched or unbranched. Preferably the alkenyl group is a C₂₋₂₀ alkenyl group, more preferably a C₂₋₁₅ alkenyl group, more preferably still a C₂₋₁₂ alkenyl group, or preferably a C₂₋₆ alkenyl group, and most preferably a C₂₋₄ alkenyl group. The term “alkenylene” should be construed accordingly.

As used herein, the term “alkynyl” refers to a carbon chain containing one or more triple bonds, which may be branched or unbranched. Preferably the alkynyl group is a C₂₋₂₀ alkynyl group, more preferably a C₂₋₁₅ alkynyl group, more preferably still a C₂₋₁₂ alkynyl group, or preferably a C₂₋₆ alkynyl group and most preferably a C₂₋₄ alkynyl group. The term “alkynylene” should be construed accordingly.

Unless otherwise specified, an alkyl, alkenyl or alkynyl group is typically unsubstituted. However, where such a group is indicated to be unsubstituted or substituted, one or more hydrogen atoms are optionally replaced by halogen atoms or sulfonic acid groups. Preferably, a substituted alkyl, alkenyl or alkynyl group has from 1 to 10 substituents, more preferably 1 to 5 substituents, more preferably still 1, 2 or 3 substituents and most preferably 1 or 2 substituents, for example 1 substituent. Preferably a substituted alkyl, alkenyl or alkynyl group carries not more than 2 sulfonic acid substituents. Halogen atoms are preferred substituents. Preferably, though, an alkyl, alkenyl or alkynyl group is unsubstituted.

In the moiety that is an alkyl, alkenyl or alkynyl group or an alkylene, alkenylene or alkynylene group, in which (a) 0, 1 or 2 carbon atoms may be replaced by groups selected from C₆₋₁₀ arylene, 5- to 10-membered heteroarylene, C₃₋₇ carbocyclylene and 5- to 10-membered heterocyclylene groups, and (b) 0, 1 or 2 —CH₂— groups may be replaced by groups selected from —O—, —S—, —S—S—, —C(O)— and —N(C₁₋₆ alkyl)- groups, a total of 0, 1 or 2 of said carbon atoms and —CH₂— groups are preferably replaced, more preferably a total of 0 or 1. Most preferably, none of the carbon atoms or —CH₂— groups is replaced.

Preferred groups for replacing a —CH₂— group are —O—, —S— and —C(O)— groups. Preferred groups for replacing a carbon atom are phenylene, 5- to 6-membered heteroarylene, C₅₋₆ carbocyclylene and 5- to 6-membered heterocyclylene groups. As used herein, the reference to “0, 1 or 2 carbon atoms” means any terminal or non-terminal carbon atom in the alkyl, alkenyl or alkynyl chain, including any hydrogen atoms attached to that carbon atom. As used herein, the reference to “0, 1 or 2 —CH₂— groups” refers to a group which does not correspond to a terminal carbon atom in the alkyl, alkenyl or alkynyl chain.

As used herein, a C₆₋₁₀ aryl group is a monocyclic or polycyclic 6- to 10-membered aromatic hydrocarbon ring system having from 6 to 10 carbon atoms. Phenyl is preferred. The term “arylene” should be construed accordingly.

As used herein, a 5- to 10-membered heteroaryl group is a monocyclic or polycyclic 5- to 10-membered aromatic ring system, such as a 5- or 6-membered ring, containing at least one heteroatom, for example 1, 2, 3 or 4 heteroatoms, selected from O, S and N. When the ring contains 4 heteroatoms these are preferably all nitrogen atoms. The term “heteroarylene” should be construed accordingly.

Examples of monocyclic heteroaryl groups include thienyl, furyl, pyrrolyl, imidazolyl, thiazolyl, isothiazolyl, pyrazolyl, oxazolyl, isoxazolyl, triazolyl, thiadiazolyl, oxadiazolyl, pyridinyl, pyridazinyl, pyrimidinyl, pyrazinyl, triazinyl and tetrazolyl groups.

Examples of polycyclic heteroaryl groups include benzothienyl, benzofuryl, benzimidazolyl, benzothiazolyl, benzisothiazolyl, benzoxazolyl, benzisoxazolyl, benztriazolyl, indolyl, isoindolyl and indazolyl groups. Preferred polycyclic groups include indolyl, isoindolyl, benzimidazolyl, indazolyl, benzofuryl, benzothienyl, benzoxazolyl, benzisoxazolyl, benzothiazolyl and benzisothiazolyl groups, more preferably benzimidazolyl, benzoxazolyl and benzothiazolyl, most preferably benzothiazolyl. However, monocyclic heteroaryl groups are preferred.

Preferably the heteroaryl group is a 5- to 6-membered heteroaryl group. Particularly preferred heteroaryl groups are thienyl, pyrrolyl, imidazolyl, thiazolyl, isothiazolyl, pyrazolyl, oxazolyl, isoxazolyl, triazolyl, pyridinyl, pyridazinyl, pyrimidinyl and pyrazinyl groups. More preferred groups are thienyl, pyridinyl, pyridazinyl, pyrimidinyl, pyrazinyl, pyrrolyl and triazinyl, most preferably pyridinyl.

As used herein, a 5- to 10-membered heterocyclyl group is a non-aromatic, saturated or unsaturated, monocyclic or polycyclic C₅₋₁₀ carbocyclic ring system in which one or more, for example 1, 2, 3 or 4, of the carbon atoms are replaced with a moiety selected from N, O, S, S(O) and S(O)₂. Preferably, the 5- to 10-membered heterocyclyl group is a 5- to 6-membered ring. The term “heterocyclyene” should be construed accordingly.

Examples of heterocyclyl groups include azetidinyl, oxetanyl, thietanyl, pyrrolidinyl, imidazolidinyl, oxazolidinyl, isoxazolidinyl, thiazolidinyl, isothiazolidinyl, tetrahydrofuranyl, tetrahydrothienyl, tetrahydropyranyl, tetrahydrothiopyranyl, dithiolanyl, dioxolanyl, pyrazolidinyl, piperidinyl, piperazinyl, hexahydropyrimidinyl, methylenedioxyphenyl, ethylenedioxyphenyl, thiomorpholinyl, S-oxo-thiomorpholinyl, S,S-dioxo-thiomorpholinyl, morpholinyl, 1,3-dioxolanyl, 1,4-dioxolanyl, trioxolanyl, trithianyl, imidazolinyl, pyranyl, pyrazolinyl, thioxolanyl, thioxothiazolidinyl, 1H-pyrazol-5-(4H)-onyl, 1,3,4-thiadiazol-2(3H)-thionyl, oxopyrrolidinyl, oxothiazolidinyl, oxopyrazolidinyl, succinimido and maleimido groups and moieties. Preferred heterocyclyl groups are pyrrolidinyl, imidazolidinyl, oxazolidinyl, isoxazolidinyl, thiazolidinyl, isothiazolidinyl, tetrahydrofuranyl, tetrahydrothienyl, tetrahydropyranyl, tetrahydrothiopyranyl, dithiolanyl, dioxolanyl, pyrazolidinyl, piperidinyl, piperazinyl, hexahydropyrimidinyl, thiomorpholinyl and morpholinyl groups and moieties. More preferred heterocyclyl groups are tetrahydropyranyl, tetrahydrothiopyranyl, thiomorpholinyl, tetrahydrofuranyl, tetrahydrothienyl, piperidinyl, morpholinyl and pyrrolidinyl groups.

For the avoidance of doubt, although the above definitions of heteroaryl and heterocyclyl groups refer to an “N” moiety which can be present in the ring, as will be evident to a skilled chemist the N atom will be protonated (or will carry a substituent as defined below) if it is attached to each of the adjacent ring atoms via a single bond.

As used herein, a C₃₋₇ carbocyclyl group is a non-aromatic saturated or unsaturated hydrocarbon ring having from 3 to 7 carbon atoms. Preferably it is a saturated or mono-unsaturated hydrocarbon ring (i.e. a cycloalkyl moiety or a cycloalkenyl moiety) having from 3 to 7 carbon atoms, more preferably having from 5 to 6 carbon atoms. Examples include cyclopropyl, cyclobutyl, cyclopentyl and cyclohexyl and their mono-unsaturated variants. Particularly preferred carbocyclic groups are cyclopentyl and cyclohexyl. The term “carbocyclylene” should be construed accordingly.

Where specified, 0, 1 or 2 carbon atoms in a carbocyclyl or heterocyclyl group may be replaced by —C(O)— groups. As used herein, the “carbon atoms” being replaced are understood to include the hydrogen atoms to which they are attached. When 1 or 2 carbon atoms are replaced, preferably two such carbon atoms are replaced. Preferred such carbocyclyl groups include a benzoquinone group and preferred such heterocyclyl groups include succinimido and maleimido groups.

Unless otherwise specified, an aryl, heteroaryl, carbocyclyl or heterocyclyl group is typically unsubstituted. However, where such a group is indicated to be unsubstituted or substituted, one or more hydrogen atoms are optionally replaced by halogen atoms or C₁₋₆ alkyl, C₁₋₆ alkoxy, C₁₋₆ alkylthiol, —N(C₁₋₆alkyl)(C₁₋₆ alkyl), nitro or sulfonic acid groups. Preferably, a substituted aryl, heteroaryl, carbocyclyl or heterocyclyl group has from 1 to 4 substituents, more preferably 1 to 2 substituents and most preferably 1 substituent. Preferably a substituted aryl, heteroaryl, carbocyclyl or heterocyclyl group carries not more than 2 nitro substituents and not more than 2 sulfonic acid substituents. Preferred substituents are halogen atoms and C₁₋₄ alkyl and C₁₋₄ alkoxy groups. Particularly preferred substituents are halogen atoms. Preferably, though, an aryl, heteroaryl, carbocyclyl or heterocyclyl group is unsubstituted.

As used herein, a C₁₋₆ alkoxy group is a C₁₋₆ alkyl (e.g. a C₁₋₄ alkyl) group which is attached to an oxygen atom.

As used herein, a C₁₋₆ alkylthiol group is a C₁₋₆ alkyl (e.g. a C₁₋₄ alkyl) group which is attached to a sulfur atom.

As used herein, a 5- to 10-membered heterocyclylthiol is a 5- to 10-membered (e.g., a 5- to 6-membered) heterocyclyl group which is attached to a sulfur atom.

As used herein, a C₆₋₁₀ arylthiol is a C₆₋₁₀ aryl (e.g., a phenyl) group which is attached to a sulfur atom.

As used herein, a C₃₋₇ carbocyclylthiol is a C₃₋₇ carbocyclyl (e.g., a C₅₋₆ carbocyclyl) group which is attached to a sulfur atom.

Where any of the groups defined herein contain or consist of hydrogen atoms, one or more of these hydrogen atoms may be replaced by deuterium atoms. Preferably when a group contains at least one deuterium atom, then all of the hydrogen atoms in that group are replaced by deuterium atoms, i.e. the group is “fully deuterated”.

As used herein, a halogen atom means a fluorine, chlorine, bromine, iodine or astatine atom. The term radioisotopic halogen atom means a halogen atom which is also a radionuclide, such as ¹⁸F, ¹²⁰I, ¹²³I, ¹²⁴I, ¹²⁵I, ¹³¹I and ²¹¹At.

As used herein, the term label means a moiety which is capable of generating a detectable signal in a test sample. In this context, test sample includes laboratory samples or a part or whole of a human or animal subject. Thus, for example, the term label encompasses moieties that can be detected when the construct to which they are attached has been administered to a human or animal subject. Labels are also commonly known in the art as “tags”, “probes” and “tracers”.

The products obtained according to the present invention contain at least one label, i.e. a radioisotopic halogen atom. In a preferred embodiment of the present invention, the products of the invention contain at least two labels, for example two labels, wherein each label is different.

Labels suitable for the triazole derivative of formula (I) include radioisotopic labels, fluorescent labels, chromogenic labels, MRI contrast agents, CT contrast agents and ultrasound contrast agents. It will be appreciated that the choice of label or labels when carrying out the present invention will be determined by the intended use of the trifunctionalised triazole derivative. In a preferred embodiment of the invention, when the product of the invention contains at least two labels, each said label belongs to a different one of the classes of label set out in this paragraph.

A radioisotopic label is a moiety that comprises or consists of a radionuclide. Examples of radionuclides include ¹⁸F, ¹²⁰I, ¹²³I, ¹²⁴I, ¹²⁵I, ¹³¹I, ²¹¹At, ²¹²Bi, ⁸⁸Y, ⁹⁰Y, ^{99m}Tc, ⁶⁰Cu, ⁶¹Cu, ⁶²Cu, ⁶⁴Cu, ⁶⁷Cu, ¹⁸⁶Re, ¹⁸⁸Re, ²²⁶Ra, ⁶⁶Ga, ⁶⁷Ga, ⁶⁸Ga, ¹¹¹In, ^{113m}In, ¹¹⁴In, ^{114m}In, ¹⁵³Sm, ¹⁰B, ³H, ¹¹C, ¹⁴C, ¹³N, ³²P, ³³P, ⁵⁵Fe, ⁵⁹Fe, ⁷⁵Br, ⁷⁶Br, ⁷⁷Br, ^{80m}Br, ⁸⁰Br, ⁸²Br and ³⁵S. Typically the radioisotopic labels envisaged in the present invention are radioisotopic labels that are suitable for use in nuclear medicine. For example, ¹²⁴I, ¹⁸F and ¹¹C are common PET labels, ^{99m}Tc, ¹²³I, ¹²⁵ I and ¹¹¹In are common SPECT isotopes and alkyne functionalised chelates of Gd have been reacted with ¹⁸F fluoroethyl azide for use in MRI.

In one embodiment, the radioisotopic label may consist of the radionuclide alone. In another embodiment, the radionuclide may be incorporated into a larger radioisotopic label, for example by direct covalent bonding to a linker group or by forming a co-ordination complex with a chelating agent as herein defined. Suitable chelating agents known in the art include DTPA (diethylenetriamine-pentaacetic anhydride), NOTA (1,4,7-triazacyclononane-N,N′,N″-triacetic acid), DOTA (1,4,7,10-tetraazacyclododecane-N,N′,N″,N′″-tetraacetic acid), TETA (1,4,8,11-tetraazacyclotetra-decane-N,N′,N″,N′″-tetraacetic acid), DTTA (N¹-(p-isothiocyanatobenzyl)-diethylene-triamine-N¹,N²,N³-tetraacetic acid), DFA (N′-[5-[[5-[[5-acetylhydroxyamino)pentyl]amino]-1,4-dioxobutyl]hydroxyamino]pentyl]-N-(5-aminopentyl)-N-hydroxybutanediamide) and HYNIC (Hydrazinonicotinamide). For the avoidance of doubt, a chelating agent that contains a radionuclide is a radioisotopic label; a chelating agent that does not contain a radionuclide is referred to herein simply as a chelating agent.

One particular interest in the present invention is in the field of nuclear imaging of molecular processes in cancer, for example in imaging of hypoxia. Details of the current state of the art as regards such techniques may be found in the review article “Nuclear imaging of molecular processes in cancer” published online on 25 Sep. 2009 in Targ Oncol (DOI 10.1007/s11523-009-01230-2) and which is herein incorporated by reference in its entirety.

A fluorescent label is a moiety which comprises a fluorophore, which is a fluorescent chemical moiety. The fluorescent label may be either an inorganic moiety such as a quantum dot or an organic moiety. Fluorescent labels include the wide range of commercially available dyes such as the ALEXA® dyes, near infrared dyes, dyes whose fluorescent properties change following particular chemical reactions and/or interactions, such as interactions with a drug, toxin or metal ion, bioluminescent groups and fluorescent proteins such as GFP (green fluorescent protein).

Examples of organic fluorescent compounds which are commonly incorporated as fluorescent labels include:
the Alexa Fluor® dye family available from Invitrogen;
cyanine and merocyanine;
the BODIPY (boron-dipyrromethene) dye family, available from Invitrogen;
the ATTO dye family manufactured by ATTO-TEC GmbH;
fluorescein and its derivatives;
rhodamine and its derivatives, for example rhodamine B;
naphthalene derivatives such as its dansyl and prodan derivatives;
pyridyloxazole, nitrobenzoxadiazole and benzoxadiazole derivatives;
coumarin and its derivatives;
pyrene derivatives; and
   Oregon green, eosin, Texas red, Cascade blue and Nile red, available from Invitrogen.

Preferred fluorescent labels for use in the present invention include fluorescein, rhodamine, coumarin, sulforhodamine 101 acid chloride (Texas Red) and dansyl. Coumarin and rhodamine and their derivatives are particularly preferred. Rhodamine B is presently a most preferred fluorescent label. Further fluorescent labels for use in the present invention include indocyanine green (“ICG”), Cy5.5 and Cy7.

A chromogenic label is a moiety which is coloured, which becomes coloured when it is incorporated into a product of the present invention, or which becomes coloured when it is incorporated into a product of the present invention and subsequently interacts with a secondary target species (for example, where the product of the present invention comprises a protein, which then interacts with another target molecule). Typically, the term “chromogenic label” refers to a group of associated atoms which can exist in at least two states of energy, a ground state of relatively low energy and an excited state to which it may be raised by the absorption of light energy from a specified region of the radiation spectrum. Often, the group of associated atoms contains delocalised electrons. Chromogenic moieties suitable for use in the present invention include conjugated moieties containing Π systems and metal complexes. Examples include porphyrins, polyenes, polyynes and polyaryls.

Preferred chromogenic moieties are

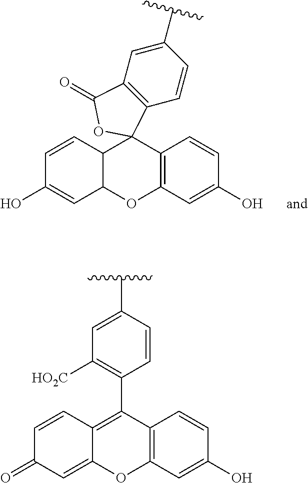 [SMILES: *c1ccc2c(c1)C(=O)OC21c2ccc(O)cc2OC2C=C(O)C=CC21 ; *c1ccc(-c2c3ccc(=O)cc-3oc3cc(O)ccc23)c(C(=O)O)c1]

As used herein, an MRI (“magnetic resonance imaging”) contrast agent means a moiety which, when incorporated into a triazole derivative in accordance with the present invention, is capable of modifying the relaxation time of, for example, a tissue and/or body cavity where it is located. As would be understood by one skilled in the art, relaxation time is a property of a substance that determines the MRI signal obtained when an MRI experiment is carried out. MRI contrast agents are commonly used in MRI to improve the visibility, for example, of internal body structures.

Many MRI contrast agents are well known. The present invention encompasses the incorporation of any MRI contrast agent as a label in the triazole derivatives. Examples of MRI contrast agents include paramagnetic gadolinium-based contrast agents, which typically comprises a gadolinium core and chelating agent, iron oxide contrast agents, chelated manganese contrast agents and barium sulphate.

A CT contrast agent is a substance that is capable of increasing the visibility of a target region of interest in a subject undergoing a CT scan. Examples of CT contrast agents include iodine-based contrast agents (e.g., diatrizoate, metrizoate, ioxaglate, iopamidol, iohexyl, ioxilan, iopromide and iodixanol) and barium-containing contrast agents.

As used herein, the term ultrasound contrast agent means a moiety which, when incorporated into a triazole derivative in accordance with the present invention, has an echogenicity that is different from the target to be imaged in an ultrasound experiment. Typically, the target is part of a human or animal body, for example an organ thereof, and the ultrasound experiment is carried out after the triazole derivative comprising the ultrasound contrast agent has been administered thereto. The presence in or around the target of the contrast agent having a markedly different echogenicity improves the overall contrast of the ultrasound image. Microbubbles are one example of an ultrasound contrast agent. The use of microbubble contrast agents is well established in the art and these agents can routinely be modified into a reagent suitable for use in creating a triazole derivative via a suitable reactive group located on the microbubble shell material.

As used herein, the term chelating agent means a substance that is capable two or more binding interactions with a target atom or ion. The use of chelating agents is very well established in the field of bioconjugates and especially in imaging applications using, for example, radioisotopic labels. Chelating agents can be routinely selected and incorporated into the products and processes of the present inventions. Specific examples of chelating agents are disclosed throughout this specification. A common type of chelating agent of particular utility in the present invention is a chelating agent capable of chelating to a metal ion, otherwise known as a “metal chelator”.

As used herein, the term biologically active moiety means a moiety that is capable of inducing a biochemical response when administered in vivo in relation to a human or animal subject. A vast range of classes of biologically active moieties have been used as one or more of the discrete moieties that together constitute a bioconjugate construct. There is of course no single structural relationship characterising such materials. A skilled person working in the field of biotechnology, and in particular with an interest in bioconjugate techniques, would immediately be aware of which substances can be regarded as being a biologically active moiety. A biologically active moiety may be a biomolecule, a fully synthetic substance, or a biomolecule that has been subjected to one or more synthetic processing techniques.

For the avoidance of doubt, it should be understood that where Y and/or Z is/are a biologically active moiety the requirement for being “capable of inducing a biochemical response when administered in vivo in relation to a human or animal subject” is met when either the biologically active moiety itself or the resulting triazole derivative, as an overall structure, has that capability. Thus, it is possible but not essential that the biologically active moiety is capable of inducing a biochemical response when it is an isolated substance, as long as the triazole derivative incorporating the biologically active moiety is capable of inducing a biochemical response.

Non-exclusive examples of a biologically active moiety include a moiety capable of binding to a biomolecule of interest in vivo, a moiety capable of modifying the physicochemical properties of the derivative in vivo, a peptide, a protein, an antibody or antibody fragment, a particle, a DNA or DNA analogue, a chemical entity that is sensitive to changes in in vivo environment and a drug moiety.

As used herein, the term “moiety capable of binding to a biomolecule of interest in vivo” means any moiety that is allows triazole derivative carrying such a moiety to be targeted at a biomolecule of interest. The concept of targeting bioconjugates to specific in vivo targets is now well established in biotechnology. The potential medical and/or diagnostic advantages of such targeting are vast.

Examples of moieties capable of binding to a biomolecule of interest in vivo are substances that bind to a site on a target protein, for example on a receptor. The interaction with the protein is typically non-covalent. For example, the interaction may be through ionic bonding, hydrogen bonding or van der Waals' interactions. However, it is also possible for to form covalent bonds to the protein. Typically, the binding moiety is capable of altering the chemical conformation of the protein when it interacts with it. The binding is typically specific, which means that the moiety has a substantially greater propensity to bind to the protein of interest than to any other substance present in the vicinity of that protein in vivo.

Examples of moieties capable of binding to a protein include substrates (which are acted upon by the enzyme upon binding, for example by taking part in a chemical reaction catalysed by the enzyme), inhibitors (which inhibit protein activity on binding), activators (which increase protein activity on binding) and neurotransmitters. The moiety capable of binding to the protein may have a structure that mimics the natural ligand that binds to the protein during natural biochemical processes in vivo.

Another specific type of moiety capable of binding to a biomolecule of interest in vivo is an antibody or antibody fragment, which, as described below, is capable of binding to a specific antigen.

The moiety capable of modifying the physicochemical properties of the derivative in vivo is typically a moiety that changes the way the triazole derivative behaves in vivo such as its solubility, lipophilicity and/or bioavailability. For example, the moiety may change the bioavailability of the derivative, which is the rate at which the triazole derivative is metabolised in vivo and/or delivered to a target region of interest. Numerous such moieties are well known in the field, particularly with regard to modifying the delivery of diagnostic and/or therapeutic agents.

One example of a moiety capable of modifying the bioavailability of the derivative in vivo is a moiety that is capable of enhancing the ability of the triazole derivative to cross the biological membrane. Such a moiety may be a “protein transduction domain” (PTD) or a small molecule carrier (“SMC” or “molecular tug”) such as those described in WO 2009/027679, the content of which is hereby incorporated by reference in its entirety.

A further example of a moiety capable of modifying the bioavailability of the derivative in vivo is a “polymeric moiety”, which is a single polymeric chain (branched or unbranched), which is derived from a corresponding single polymeric molecule. Polymeric moieties may be natural polymers or synthetic polymers.

As is well known in the biochemical field, creation of bioconjugate constructs comprising a polymeric moiety is useful in many in vivo applications. For example, various properties of a macromolecule such as a triazole derivative bearing a label can be modified by attaching a polymeric moiety thereto, including solubility properties, surface characteristics and stability in solution or on freezing. Another common application involves conjugating the polymeric moiety to a moiety of biological or diagnostic significance with the aim of enhancing biocompatibility, reducing or eliminating immune response on administration, and/or increasing in vivo stability.

The nature of the polymeric moiety of course depends upon the intended use of the triazole derivative. Exemplary polymeric moieties for use in accordance with the present invention include polysaccharides, polyethers, polyamino acids (such as polylysine), polyvinyl alcohols, polyvinylpyrrolidinones, poly(meth)acrylic acid and derivatives thereof, polyurethanes and polyphosphazenes. Typically such polymers contain at least ten monomeric units. Thus, for example, a polysaccharide typically comprises at least ten monosaccharide units.

Two particularly preferred polymeric molecules are dextran and polyethylene glycol (“PEG”), as well as derivatives of these molecules (such as monomethoxypolyethylene glycol, “mPEG”). Preferably, the PEG or derivative thereof has a molecular weight of less than 20,000. Preferably, the dextran or derivative thereof has a molecular weight of 10,000 to 500,000.

As used herein, the terms “peptide” and “protein” mean a polymeric moiety made up of amino acid residues. As a person of skill in the art will be aware, the term “peptide” is typically used in the art to denote a polymer of relatively short length and the term “protein” is typically used in the art to denote a polymer of relatively long length. As used herein, the convention is that a peptide comprises up to 50 amino acid residues whereas a protein comprises more than 50 amino acids. However, it will be appreciated that this distinction is not critical since the functional moieties identified in the present application can typically represent either a peptide or a protein.

As used herein, the term “polypeptide” is used interchangeable with “protein”.

As used herein, a peptide or a protein can comprise any natural or non-natural amino acids. For example, a peptide or a protein may contain only α-amino acid residues, for example corresponding to natural α-amino acids. Alternatively the peptide or protein may additionally comprise one or more chemical modifications. For example, the chemical modification may correspond to a post-translation modification, which is a modification that occurs to a protein in vivo following its translation, such as an acylation (for example, an acetylation), an alkylation (for example, a methylation), an amidation, a biotinylation, a formylation, glycosylation, a glycation, a hydroxylation, an iodination, an oxidation, a sulfation or a phosphorylation. A person of skill in the art would of course recognise that such post-translationally modified peptides or proteins still constitute a “peptide” or a “protein” within the meaning of the present invention. For example, it is well established in the art that a glycoprotein (a protein that carries one or more oligosaccharide side chains) is a type of protein.

As used herein, the term “antibody or antibody fragment” means a protein that is capable of binding to a specific antigen via an epitope on the antigen, or a fragment of such a protein. Antibodies include monoclonal antibodies and polyclonal antibodies. Monoclonal antibodies are preferred.

As used herein, the term DNA or DNA analogue includes deoxyribonucleic acids made up of one or more nucleotides, which may be single stranded or double stranded. The term DNA analogue includes RNAs. DNA analogues may also be DNA or RNA molecules which have been chemically modified, for example by incorporating one or more non-natural bases and/or base pair mismatches and/or synthetic side chains and/or end groups.

As used herein, the term “chemical entity that is sensitive to changes in in vivo environment” means a moiety that undergoes a physical and/or chemical (preferably chemical) change when the triazole derivative is subjected to specific environmental conditions in vivo, but does not undergo such a change when subjected to other specific environment conditions in vivo. Examples of such chemical entities include:
a redox active group which is capable of engaging in a redox reaction when triazole derivative encounters another redox active group such that a redox pair is formed;
a radical trapping moiety which is capable of trapping free radicals;
a pH sensitive group that undergoes a chemical change under certain pH conditions and either no change or a different change under other pH conditions.

It will be seen that the chemical entity can take various forms. Therefore the reference to environmental conditions should be interpreted accordingly, and can embrace any chemical conditions relating to the chemical substances in the vicinity of the chemical entity, including the presence or absence of solvents, reactive compounds, free radicals, acids and/or bases and so on, as well as physical environmental conditions such as temperature and pressure.

As used herein, a drug moiety means a moiety that is capable of modifying body function when administered in vivo. Preferably the drug moiety is capable of causing a therapeutic effect when administered in vivo. Any drug moiety located the triazole derivatives of the invention can correspond to a known drug molecule that is attached to the triazole derivative via a suitable point of attachment on the drug.

Drugs that can be used as the drug moiety in the present invention include radioisotopic therapeutic agents, cytotoxic agents such as doxorubicin, methotrexate and derivatives thereof, cytotoxin precursors which are capable of metabolising in vivo to produce a cytotoxic agent, anti-neoplastic agents, anti-hypertensives, cardioprotective agents, anti-arrhythmics, ACE inhibitors, anti-inflammatories, diuretics, muscle relaxants, local anaesthetics, hormones, cholesterol lowering drugs, anti-coagulants, anti-depressants, tranquilizers, neuroleptics, analgesics such as a narcotic or anti-pyretic analgesics, anti-virals, anti-bacterials, anti-fungals, bacteriostats, CNS active agents, anti-convulsants, anxiolytics, antacids, narcotics, antibiotics, respiratory agents, anti-histamines, immunosuppressants, immunoactivating agents, nutritional additives, anti-tussives, diagnostic agents, emetics and anti-emetics, carbohydrates, glycosoaminoglycans, glycoproteins and polysaccharides, lipids, for example phosphatidyl-ethanolamine, phosphtidylserine and derivatives thereof, sphingosine, steroids, vitamins, antibiotics, including lantibiotics, bacteristatic and bactericidal agents, antifungal, anthelminthic and other agents effective against infective agents including unicellular pathogens, small effector molecules such as noradrenalin, alpha adrenergic receptor ligands, dopamine receptor ligands, histamine receptor ligands, GABA/benzodiazepine receptor ligands, serotonin receptor ligands, leukotrienes and triodothyronine, and derivatives thereof.

Preferably the drug moiety is a radioisotopic therapeutic agent, which is an agent that is radioisotopically unstable and is capable of destroying undesirable material in the body when it decays. Examples of such agents include ¹³¹I either alone (for example, for treating thyroid cancer) or incorporated into an organic compound (for example, in metaiodobenzylguanidine for treating neuroblastoma), hormone-bound ¹⁷⁷Lt and ⁹⁰Y (for treating neuroendocrine tumours) and isotopes such as ⁸⁹Sr and ¹⁵³Sm ethylene diamine tetramethylene phosphonate for treatment of bone metastasis from cancer. It will be appreciated that incorporation of suitable radioisotopic therapeutic agents into the triazole derivative of the present invention can enable them to be targeted directly to regions of interest in the body.

As used herein a moiety enabling purification of the derivative means a moiety that can be used to assist a skilled person to purify the triazole derivative from a medium in which it has been placed.

Methods for purifying bioconjugates are well known in the art. For example, the moiety enabling purification may be a substance known as an “affinity tag”, which is a chemical moiety capable of interacting with an “affinity partner” when both the affinity tag and the affinity partner are present in a single sample. Typically, the affinity tag is capable of forming a specific binding interaction with the affinity partner. A specific binding interaction is a binding interaction which is stronger than any binding interaction that may occur between the affinity partner and any other chemical substance present in a sample. A specific binding interaction may occur, for example, between an enzyme and its substrate.

One affinity tag/affinity partner pair that is particularly widely used in biochemistry is the biotin/(strept)avidin pair. Avidin and streptavidin are proteins which can be used as affinity partners for binding with high affinity and specificity to an affinity tag derived from biotin (5-[(3aS,4S,6aR)-2-oxohexahydro-1H-thieno[3,4-d]imidazol-4-yl]pentanoic acid). Other affinity tag/affinity partner pairs commonly used in the art include amylase/maltose binding protein, glutathione/glutathione-S-transferase and metal (for example, nickel or cobalt)/poly(His). As one of skill in the art would appreciate, either member of the pair could function as the “affinity tag”, with the other member of the pair functioning as the “affinity partner”. The terms “affinity tag” and “affinity partner” are thus interchangeable.

As used herein, the term reactive linker means a group which is capable of linking one discrete chemical moiety to another. The nature of the reactive linkers used in accordance with the present invention is not important. A person of skill in the art would recognise that reactive linkers are routinely used in the construction of bioconjugate molecules. Typically, a reactive linker for use in the present invention is an organic group. Typically, such a reactive linker has a molecular weight of 50 to 1000, preferably 100 to 500. Examples of reactive linkers appropriate for use in accordance with the present invention are common general knowledge in the art and described in standard reference text books such as “Bioconjugate Techniques” (Greg T. Hermanson, Academic Press Inc., 1996), the content of which is herein incorporated by reference in its entirety.

As used herein, electrophilic leaving group means a substituent attached to a chemical moiety which can be displaced by a nucleophilic attack with the result that a new chemical bond is created from the attacking species to the chemical moiety. Those of skill in the art are routinely able to select electrophilic leaving groups that would be suitable for locating on a particular compound and for reacting with a particular nucleophile.

As used herein, the expression “in the presence of Cu(II) ions” in relation to a process of the invention means that the process comprises adding Cu(II) ions to the reaction medium and/or adding one or more reagents to the reaction medium whereby Cu(II) ions are formed in situ (for example, by adding Cu(I) ions together with an oxidising agent that is capable of oxidising the Cu(I) ions to Cu(II) ions in situ). The term “in the presence of Cu(I) ions” means that the process comprises adding Cu(I) ions to the reaction medium. Typically the term “in the presence of Cu(II) ions” is synonymous with “catalysed by Cu(II) ions”.

As used herein, a base means a substance which is a Lewis base and/or a Brønsted base. Preferably the base is a Brønsted base, which is a substance that is capable of accepting protons.

As used herein, the expression “in the presence of a Cu(II) salt” in relation to a process of the invention means that the process comprises adding a Cu(II) salt to the reaction medium. In particular, for the avoidance of doubt, the expression “in the presence of a Cu(II) salt” does not necessitate that the Cu(II) ions remain bonded or otherwise associated with their respective counter ions once they are in the reaction medium.

As used herein, the term “Cu(II) carboxylic acid salt” means a salt containing a Cu(II) centre ion and two carboxylate ions, which may be the same or different. Preferably the two carboxylate ions are the same. Examples of suitable carboxylate ions include methanoate and ethanoate ions.

As used herein, the term “ligand capable of co-ordinating to Cu(II) ions” means any ligand that is capable of forming a co-ordination complex with Cu(II) ions in the reaction medium. It is possible for the ligand capable of co-ordinating to Cu(II) ions to be the same substance as the base and/or any solvent used in the process of the invention. For example, if triethylamine is used as the base then this substance also inherently functions as a ligand capable of co-ordinating to Cu(II) ions.

The present invention provides a process for producing a triazole derivative represented by the formula (I)

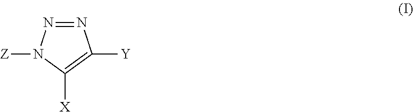

The claimed process involves a reaction (B) between a disubstituted alkyne of formula (II′) and an azide of formula (III) and is carried out in the presence of (is catalysed by) Cu(II) ions, with a base also being present.

The process of the present invention can be, and preferably is, carried out in an aqueous solution, i.e. in the presence of water. This is an advantage compared to previously known coupling reactions to produce triazole derivatives, which have been carried out under strictly anhydrous conditions.

This process can optionally be carried out using the alkyne of formula (II′) as the starting material. However, preferably, the alkyne of formula (II) is itself obtained by (A) reacting a reacting a terminal alkyne represented by the formula (II)

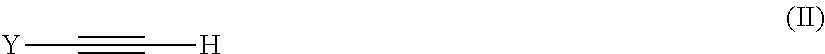

with a radioisotopic halogen anion, in the presence of Cu(II) ions and a base.

The same reaction conditions can be used for carrying out (A) and (B). In particular both reactions are catalysed by the Cu(II) ions in the presence of a base. Therefore, it is usually convenient to obtain the alkyne represented by the formula (II′) and to react this alkyne represented by the formula (II′) in a single synthetic procedure that is carried out without isolating the alkyne represented by the formula (II′) from the reaction medium. In this embodiment, therefore, the overall reaction can be seen to be a three-component reaction between (i) a terminal alkyne represented by the formula (II), (ii) a radioisotopic halogen anion and (iii) an azide represented by the formula (III). Thus, the present invention also provides a process for producing a triazole derivative represented by the formula (I)

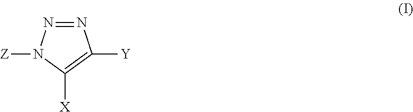

which process comprises reacting a terminal alkyne represented by the formula (II)

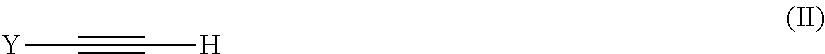

with an azide represented by the formula (III)

Z—N₃ (III)

and a radioisotopic halogen anion;
said process being carried out in the presence of Cu(II) ions and a base.

The present inventors have found that the derivative of formula (I) can advantageously be prepared by reacting these three components together in the presence of Cu(II) ions and a base. It has in particular been found that under these conditions good yields of the three-component reaction are possible over the competing two-component reaction between only the terminal alkyne and the azide (which yields a product that is analogous to that represented by the formula (I), but which carries a hydrogen atom instead of the group X at the triazole's 5-position). Although it is not clear why the three-component reaction proceeds favourably under these conditions, it appears that the Cu(II) catalyst enhances the selectivity of the reaction compared to a Cu(I) catalyst as utilised in the prior art methods of Li et al (J Org Chem, 2008 83 3630-3633) and Wu et al (Synthesis 2005 1314).

It will be appreciated that when X is a particular halogen atom, the reagent used to prepare the alkyne represented by the formula (II′) is the corresponding halogen anion (for example, if X is I then the component (c) is I′). More specifically, X is a radioisotopic halogen atom. Radioisotopic halogen atoms can be used as labels in medical imaging methods and their nuclear decay can also be exploited in methods of 120% radiotherapy. Preferred radioisotopic halogen atoms are ¹²⁰I, ¹²³I (which is used, for example, in SPECT imaging), ¹²⁴I (used, for example, in PET imaging), ¹²⁵I, ¹³¹I (which is used, for example, in radiotherapeutic thyroid ablation) and ²¹¹At (used, for example, in radiotherapy). More preferably the radioisotopic halogen atom is selected from ¹²⁰I, ¹²³I, ¹²⁴I, ¹²⁵I and ¹³¹I. Most preferably the radioisotopic halogen atom is selected from ¹²³I, ¹²⁴I, ¹²⁵I and ¹³¹I.

For the avoidance of doubt, as X is a radioisotopic halogen atom, it can be regarded as being a “label”. This applies even in circumstances when X can also be regarded as being a “drug moiety”, for example when it is ¹³¹I or ²¹¹At, since radioisotopic drug moieties are generally simultaneously capable of being detected.

Preferably two of X, Y and Z represent labels which are different from one another.

In a preferred embodiment, Y and Z are independently selected from a label, a chelating agent, a biologically active moiety and a moiety enabling purification of the derivative.

As explained above, if one or more reactive linkers are present in the derivative of formula (I), then their chemical structure is not important: it is only important that each reactive linker is capable of linking one discrete chemical moiety to another, Nonetheless, each reactive linker, if present, is preferably the same or different and is a moiety of formula -A(B)_{c}, wherein:
A is a linker moiety;
each B is the same or different and represents a reactive group attached to A and which is capable of linking the triazole derivative represented by the formula (I) to a label, a chelating agent, a biologically active moiety or a moiety enabling purification of the derivative; and
c is 1, 2 or 3.

A is preferably a C₁₋₂₀ alkylene group, a C₂₋₂₀ alkenylene group or a C₂₋₂₀ alkynylene group, which is unsubstituted or substituted by one or more substituents selected from halogen atoms and sulfonic acid groups, and in which (a) 0, 1 or 2 carbon atoms are replaced by groups selected from C₆₋₁₀ arylene, 5- to 10-membered heteroarylene, C₃₋₇ carbocyclylene and 5- to 10-membered heterocyclylene groups, and (b) 0, 1 or 2 —CH₂— groups are replaced by groups selected from —O—, —S—, —S—S—, —C(O)— and —N(C₁₋₆ alkyl)- groups, wherein:
(i) said arylene, heteroarylene, carbocyclylene and heterocyclylene groups are unsubstituted or substituted by one or more substituents selected from halogen atoms and C₁₋₆ alkyl, C₁₋₆ alkoxy, C₁₋₆ alkylthiol, —N(C₁₋₆ alkyl)(C₁₋₆ nitro and sulfonic acid groups; and
(ii) 0, 1 or 2 carbon atoms in said carbocyclylene and heterocyclylene groups are replaced by —C(O)— groups.

More preferably, A is an unsubstituted C₁₋₆ alkylene group, C₂₋₆ alkenylene group or C₂₋₆ alkynylene group, in which (a) 0 or 1 carbon atom is replaced by a group selected from phenylene, 5- to 6-membered heteroarylene, C₅₋₆ carbocyclylene and 5- to 6-membered heterocyclylene groups, wherein said phenylene, heteroarylene, carbocyclylene and heterocyclylene groups are unsubstituted or substituted by one or two substituents selected from halogen atoms and C₁₋₄ alkyl and C₁₋₄ alkoxy groups, and (b) 0, 1 or 2 —CH₂— groups are replaced by groups selected from —O—, —S— and —C(O)— groups.

Most preferably, A is an unsubstituted C₁₋₄ alkylene group, in which 0 or 1 carbon atom is replaced by an unsubstituted phenylene group.

As those of skill in the art would understand, the nature of the reactive group(s) B is not important. A very wide range of reactive groups are now routinely used in the art to connect together the discrete moieties that together constitute a bioconjugate construct. Such reactive groups may be capable, for example, of attaching an amine compound, a thiol compound, a carboxyl compound, a hydroxyl compound, a carbonyl compound or a compound containing a reactive hydrogen, to a cross-linker. Those of skill in the art would immediately recognise that any such reactive group would be suitable for use in accordance with the present invention. One of skill in the art would in particular be able to select an appropriate reactive group from common general knowledge, with reference to standard text books such as “Bioconjugate Techniques” (Greg T. Hermanson, Academic Press Inc., 1996), the content of which is herein incorporated by reference in its entirety.

Nonetheless, B is preferably:
(a) a group of formula -LG, —C(O)-LG, —C(S)-LG or —C(NH)-LG wherein LG is an electrophilic leaving group;
(b) a nucleophile Nu′ selected from —OH, —SH, —NH₂, —NH(C₁₋₆ alkyl) and —C(O)NHNH₂ groups;
(c) a cyclic moiety Cyc, which is capable of a ring-opening electrophilic reaction with a nucleophile;
(d) a group of formula —S(O₂)(Hal), wherein Hal is a halogen atom;
(e) a group of formula —N═C═O or —N═C═S;
(f) a group of formula —S—S(IG′) wherein IG′ represents a group of formula IG as defined herein;
(g) a group AH, which is a C₆₋₁₀ aryl group that is substituted by one or more halogen atoms;
(h) a photoreactive group capable of being activated by exposure to ultraviolet light;
(i) a group of formula —C(O)H or —C(O)(C₁₋₆ alkyl);
(j) a maleimido group;
(k) a group of formula —C(O)CHCH₂;
(l) a group of formula —C(O)C(N₂)H or -PhN₂⁺, where Ph represents a phenyl group; or
(m) an epoxide group.

Most preferably, B is selected from:
(a) groups of formula -LG, —C(O)-LG and —C(S)-LG, wherein LG is selected from halogen atoms and —O(C₁₋₆ alkyl), —SH, —S(C₁₋₆ alkyl), triflate, tosylate, mesylate, N-hydroxysuccinimidyl and N-hydroxysulfosuccinimidyl groups;
(b) groups of formula —OH, —SH and —NH₂;

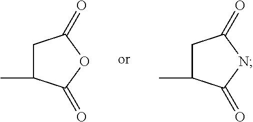

(c) a group of formula and
(d) a maleimido group.

LG is preferably selected from halogen atoms and —O(IG′), —SH, —S(IG′), —NH₂, NH(IG′), —N(IG′)(IG″), —N₃, triflate, tosylate, mesylate, N-hydroxysuccinimidyl, N-hydroxysulfosuccinimidyl, imidazolyl and azide groups, wherein IG′ and IG″ are the same or different and each represents a group of formula IG.

Nu′ is preferably selected from —OH, —SH and —NH₂ groups.

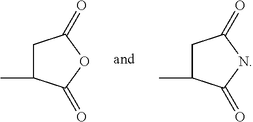

Cyc is preferably selected from the groups

Hal is preferably a chlorine atom.

AH is preferably a phenyl group that is substituted by at least one fluorine atom.

The photoreactive group is preferably selected from:
(a) a C₆₋₁₀ aryl group which is substituted by at least one group of formula —N₃ and which is optionally further substituted by one or more halogen atoms;
(b) a benzophenone group;
(c) a group of formula —C(O)C(N₂)CF₃; and
(d) a group of formula -PhC(N₂)CF₃, wherein Ph represents a phenyl group.
   c is preferably 1 or 2, and most preferably 1.

As defined herein, IG is a C₁₋₂₀ alkyl group, a C₂₋₂₀ alkenyl group or a C₂₋₂₀ alkynyl group, which is unsubstituted or substituted by one or more substituents selected from halogen atoms and sulfonic acid groups, and in which (a) 0, 1 or 2 carbon atoms are replaced by groups selected from C₆₋₁₀ arylene, 5- to 10-membered heteroarylene, C₃₋₇ carbocyclylene and 5- to 10-membered heterocyclylene groups, and (b) 0, 1 or 2 —CH₂— groups are replaced by groups selected from —O—, —S—, —S—S—, —C(O)— and —N(C₁₋₆ alkyl)- groups, wherein:
(i) said arylene, heteroarylene, carbocyclylene and heterocyclylene groups are unsubstituted or substituted by one or more substituents selected from halogen atoms and C₁₋₆ alkyl, C₁₋₆ alkoxy, C₁₋₆ alkylthiol, —N(C₁₋₆ alkyl)(C₁₋₆ alkyl), nitro and sulfonic acid groups; and
(ii) 0, 1 or 2 carbon atoms in said carbocyclylene and heterocyclylene groups are replaced by —C(O)— groups.

Preferably, IG represents a moiety which is an unsubstituted C₁₋₆ alkyl group, C₂₋₆ alkenyl group or C₂₋₆ alkynyl group, in which (a) 0 or 1 carbon atom is replaced by a group selected from phenylene, 5- to 6-membered heteroarylene, C₅₋₆ carbocyclylene and 5- to 6-membered heterocyclylene groups, wherein said phenylene, heteroarylene, carbocyclylene and heterocyclylene groups are unsubstituted or substituted by one or two substituents selected from halogen atoms and C₁₋₄ alkyl and C₁₋₄ alkoxy groups, and (b) 0, 1 or 2 —CH₂— groups are replaced by groups selected from —O—, —S— and —C(O)— groups.

More preferably, IG represents a moiety which is an unsubstituted C₁₋₆ alkyl group, in which (a) 0 or 1 carbon atom is replaced by a group selected from unsubstituted phenylene, 5- to 6-membered heteroarylene, C₅₋₆ carbocyclylene and 5- to 6-membered heterocyclylene groups.

Most preferably, IG represents an unsubstituted C₁₋₆ alkyl group.

The process is preferably carried out using non-carried added radioisotopic halogen anions, for example in salt form such as the sodium salt. Preferably the radioisotopic halogen anions are supplied in an aqueous solution. The process is therefore typically carried out in the presence of water. Typically the concentration of radioisotopic halogen anions used in the process is at most 10 nM (nanomolar), preferably at most 1 nM.

The process of the present invention is particular advantageous when the process is carried out using radioisotopic halogen anions in an aqueous solution. All previous reactions leading to formation of a 1,4-substituted, 5-halo-1,2,3-triazole have used a source of Cu(I) ions and have relied on the reaction being carried out under strictly non-aqueous conditions. For example, Wu et al (Synthesis 2005 1314) used ICI (a source of I⁺ cations) with triethylamine and a CuI catalyst under anhydrous conditions aimed at preventing the competing process for forming a non-iodinated 1,4-substituted-1,2,3-triazole. It would be envisaged that were water present the reactive intermediate would simply abstract a proton from the reaction medium rather than react with the iodine reagent to form the desired trisubstituted product. Similarly, Li et al (J Org Chem 2008 83 3630-3633) proposed a reaction carried out under anhydrous conditions using an excess of CuI together with an oxidising agent such as NIS or NBS. Again it was necessary to exclude water to prevent the competing two-component reaction between the alkyne and the azide reagents from dominating.

It is also clear that neither the Li et al nor Wu et al procedures would be suitable when dealing with the tiny relative concentrations of halogen reagent applicable when dealing with pure radioisotopic (non-carried added) halogen sources. Typical halogen ion concentrations under these circumstances are of the order of picomolar to nanomolar. Therefore under the procedures taught in Li et al and Wu et al where the bimolecular reaction already strongly competes with the desired three-component reaction, it would be impossible to obtain three-component product when the third component, the halogen source, is present in such minute quantities.

In contrast to these prior art methods, the present inventors have surprisingly found a process using Cu(II) ions and a base in which the reaction proceeds with good yield even when dealing with radioisotopic halogen sources and even when this reagent is supplied in an aqueous solution. It appears that the catalytic reaction only proceeds in the presence of the radioisotopic halogen anion, thereby suppressing the two-component (alkyne/azide) reaction in favour of the desired three component (alkyne/azide/halogen) reaction. Accordingly, it becomes possible to introduce radioisotopic halogen atoms into the triazole product. Furthermore, the reaction does not necessitate complex preliminary steps of removing water from commercially available radioisotopic halogen anion sources: the aqueous solution can simply be used directly in the process of the invention. This means that the claimed process can easily be carried out using commercially available reagents and without any special or difficult process steps. It is therefore ideally suited for use by radiopharmacies and be easily scaled up if desired.

A further advantages of the process of the present invention is that it occurs rapidly under very mild (e.g., ambient) conditions (acceptable yields are generally obtained within approximately one hour and often substantially faster, for example within half an hour). In contrast, the reaction times reported in the literature are typically from one to five hours.

In an exemplary aspect, the process of the present invention is a process for producing a triazole derivative represented by the formula (I)

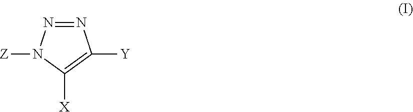

wherein:
X represents a radioisotopic halogen atom; and
Y and Z each represent, independently of one another, a moiety which is either:
   a) a label, a chelating agent, a biologically active moiety or a moiety enabling purification of the derivative; or
   b) a reactive linker for linking the triazole derivative represented by the formula (I) to a label, a chelating agent, a biologically active moiety, or a moiety enabling purification of the derivative;
      which process comprises reacting a terminal alkyne represented by the formula (II)

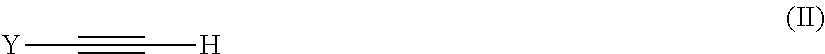

with an azide represented by the formula (III)

Z—N₃ (III)

and a radioisotopic halogen anion;
said process being carried out in the presence of Cu(II) ions, a base and water.

The following paragraphs explain preferred reaction conditions under which the process of the present invention may be carried out. For the avoidance of doubt, it should be noted that the same preferred reaction conditions apply in relation to both (A) and (B). Therefore references below to the “reaction” apply equally to (A) and (B), either individually or when they are carried out together in a single reaction medium.

Preferably, the reaction is carried out in the presence of a Cu(II) salt. Preferably the Cu(II) salt is selected from CuCl₂, CuBr₂, CuSO₄, a Cu(II) carboxylic acid salt and Cu(II) oxalate. More preferably the Cu(II) salt is selected from CuCl₂ and a Cu(II) carboxylic acid salt. Most preferably the Cu(II) salt is selected from CuCl₂ and Cu(II) acetate.

In an alternative preferred aspect, the reaction is carried out in the presence of an oxidising agent capable of oxidising Cu(I) ions to Cu(II) ions in situ. The oxidising agent capable of oxidising Cu(I) ions to Cu(II) ions in situ is preferably selected from N-iodosuccinimide, N-bromosuccinimide, quinine, H₂O₂ and FeCl₃, with N-iodosuccinimide and N-bromosuccinimide being preferred and N-iodosuccinimide being particularly preferred.

The reaction may be carried out in the presence of Cu(I) ions (as well as in the presence of Cu(II) ions, the latter being essential).

In the process of the present invention, the base can be any base that is capable of acting as a proton acceptor. Numerous bases have been used in conventional “click reactions” and would therefore be well known to the person skilled in the art: any such base could be utilised in accordance with the present invention. The base may be the solvent itself and/or simultaneously be capable of acting as a ligand capable of co-ordinating to Cu(II) ions.

Suitable bases include bases that contain amine groups, heterocyclic nitrogen-containing basic compounds (e.g., histidine-, pyridine- and quinoline-based compounds), guanidines, alkoxy (e.g., C₁₋₆ alkoxy) ions, phenoxy ions, acetate ions and other salts of carboxylic acids.

Preferably the base is a base that contains an amine group or a histidine derivative. Examples are histidine derivatives of formula

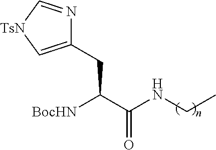

wherein n is from 1 to 20 and preferably from 3 to 12 and alkyl amines of formula (Alk₁)(Alk₂)(Alk₃)N wherein Alk₁, Alk₂ and Alk₃ are the same or different and are selected from hydrogen and alkyl groups such as C₁₋₆ alkyl groups. A particularly preferred base is triethylamine.

Preferably the base is used in an amount of at least one mole per one mole of Cu(II) ions. Preferably the base is used in an amount of at most three moles per one mole of Cu(II) ions, more preferably at most two and a half moles per one mole of Cu(II) ions and most preferably not more than two moles per one mole of Cu(II) ions. Therefore it is preferred that the base is used in an amount of from one to three moles per one mole of Cu(II) ions, preferably from one to two and a half, still more preferably from one and a half to two and a half and most preferably from one and a half to two.

Preferably the terminal alkyne represented by the formula (II) is mixed with the Cu(II) ions before adding the halogen ions. More preferably at least the terminal alkyne and the base are mixed with the Cu(II) ions before adding the halogen ions. The present inventors have found that by mixing at least the alkyne and the Cu(II) ions before adding the halogen ions the yield of the reaction and/or the reaction rate surprisingly increases. It is believed that this effect may be attributable to the alkyne and the Cu(II) ions being able to form an initial co-ordination complex that can act as a reaction template before the system becomes activated for reaction by addition of the halogen ions.

Preferably the process of the invention is carried out in the presence of a ligand capable of co-ordinating to Cu(II) ions. A person skilled in the art will be familiar with the broad range of ligands that are commonly used in classical “click reactions” and the same ligands can be employed in the present invention. A skilled person would therefore be able to select suitable ligands as a matter of routine. Examples of suitable ligands and those containing amine groups and/or phosphine groups. At present, a preferred such ligand is triethylamine, which can simultaneously function as the base in the reaction. Another preferred ligand is a histidine derivative having the formula

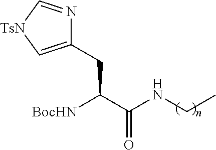

wherein n is from 1 to 20 and preferably from 3 to 12.

In the process of the invention the reaction is typically carried out in the liquid phase. Normally one or more solvents are present. The identity of the solvent or solvents has not been found to be important. As noted above, when radioisotopic halogen anions are used these are typically provided in aqueous solution and therefore the reaction medium in that case comprises water. One or more non-aqueous solvents can also be present, for example DMF, DMSO, CH₃CN and THF. At present a preferred non-aqueous solvent is CH₃CN.

The process can be carried out at room temperature. Typically the temperature is from 2 to 95° C., such as from 10 to 60° C., preferably from 10 to 50° C., more preferably from 15 to 30° C. In another preferred embodiment, the temperature is from 20 to 60° C.

Although in one preferred aspect of the invention each of Y and Z is selected from a label, a chelating agent a biologically active moiety, or a moiety enabling purification of the derivative, it is of course also possible that at least one of Y and Z represents a reactive linker. In that case, the derivative represented by the formula (I) is suitable for being functionalised by derivitisation of the reactive linker(s). Typically each such linker will be derivitised to convert Y and/or Z into a label, a chelating agent, a biologically active moiety, or a moiety enabling purification of the derivative.

A skilled person would understand that, depending on the nature of the label, chelating agent, biologically active moiety, or moiety enabling purification of the derivative, it may sometimes be desirable to produce the final multi-functionalised derivative via such a multi-step procedure. For example, if it is intended to attach the triazole to a complex biological substance such as a protein (e.g., an antibody), then it may be desirable to carry out the process of the invention in such a way as to produce a triazole derivative carrying a reactive linker suitable for linking the triazole to this biological substance in a second synthetic step.

Therefore, in a further preferred aspect of the present invention, when at least one of Y and Z represents a reactive linker said process further comprises linking, independently, each said reactive linker to a label, a chelating agent, a biologically active moiety, or a moiety enabling purification of the derivative. Clearly one or more linking steps may be required, depending on how many of the groups Y and Z represent a reactive linker. Preferably at most one of Y and Z represents a reactive linker.

It will also be appreciated that one reactive linker may itself comprises more than one reactive site and thus be capable of linking to more than one label, chelating agent, biologically active moiety, and/or moiety enabling purification of the derivative.

The nature of the one or more steps of linking a reactive linker to a label, a chelating agent, a biologically active moiety, or a moiety enabling purification of the derivative will of course be determined by the identity of the reactive linker and the group to be linked. A skilled worker in this filed would be routinely capable of selecting appropriate reaction conditions for carrying out any such linking steps, using common general knowledge in the art, supplemented, if necessary, by referring to routine procedures described in text books such as “Bioconjugate Techniques” (Greg T. Hermanson, Academic Press Inc., 1996), the content of which is herein incorporated by reference in its entirety.

The present invention also provides a triazole derivative represented by the formula (I). The groups X, Y and Z, and preferred aspects thereof, are as defined in above in relation to the process of the present invention for producing such a triazole derivative. Furthermore, in this product Y and Z each represent, independently of one another, a label, a chelating agent, a biologically active moiety, or a moiety enabling purification of the derivative.

Thus, this product is a triazole derivative represented by the formula (I)

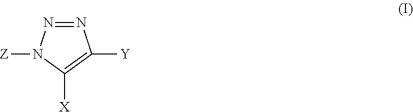

wherein:
X represents a radioisotopic halogen atom; and
Y and Z each represent, independently of one another, a label, a chelating agent, a biologically active moiety, or a moiety enabling purification of the derivative.

The present invention further provides a triazole derivative for use in a diagnostic method practised on the human or animal body. In this embodiment, the diagnostic method preferably comprises a method of medical imaging. The method of medical imaging is preferably selected from imaging with PET, SPECT, MRI, CT, ultrasound and optical techniques. Preferably the diagnostic method is practised on the human body.

The diagnostic methods specified herein are all well known and established in the diagnostic field. PET is an acronym for positron emission tomography. SPECT is an acronym for single photon emission computed tomography. MRI is an acronym for magnetic resonance imaging. CT is an acronym for computed tomography.

Clearly in this embodiment the triazole derivative must be a substance of diagnostic relevance to the particular diagnostic method being carried out. At least one of X, Y and Z is a label which is susceptible to detection using the diagnostic method. Preferably the label is susceptible to detection using one or more techniques selected from PET, SPECT, MRI, CT, ultrasound and optical techniques.

It will be appreciated that, as X is a radioisotopic halogen atom, X is necessarily a label.

Preferably two of the groups X, Y and Z are labels. Preferably in this aspect the two labels are different labels that are susceptible to detection using different techniques. For example, each label may be susceptible to detection by a different technique selected from PET, SPECT, MRI, CT, ultrasound and optical techniques. In this aspect a single triazole derivative can be used as the diagnostic agent in two complementary diagnostic methods.

The present invention also provides a method of imaging. The method is preferably selected from imaging with PET, SPECT, MRI, CT, ultrasound and optical techniques. Preferably the method is practised on the human body. The triazole derivative is as defined for the triazole derivative for use in a diagnostic method practised on the human or animal body.

In a preferred aspect of the imaging method of the invention, the method further comprises a step of administering said triazole derivative to the human or animal subject prior to said step of measuring the distribution in vivo of said triazole derivative. Preferably the administration does not involve a surgical step. For example, the administration may consist of orally administering the triazole derivative.

The method of imaging of the present invention generally results only in a measurement of the triazole derivative and it does not therefore involve comparing the measured distribution with standard values, finding a significant deviation and attributing that deviation to a particular clinical picture. However, in one embodiment the method does further comprise comparing the measured distribution with standard values, finding a significant deviation and attributing that deviation to a particular clinical picture. Preferably at least one and most preferably all of the steps of comparing the measured distribution with standard values, finding a significant deviation and attributing that deviation to a particular clinical picture are carried out without the presence of the body, for example they may be carried out on a computer.

The present invention still further provides a triazole derivative for use in a method of treatment of the human or animal body. In this embodiment, at least one of X, Y and Z is a drug moiety.

The method of treatment is preferably radiotherapy. For example, the triazole derivative may comprise a radioisotopic substance that decays in vivo to destroy target tissue material such as a tumour. However, it will also be appreciated that the method of treatment may be a non-radiotherapeutic method. For example, one of X, Y and Z may be a drug moiety such as those herein described for treating a particular disease state.

Treatment of cancer by radiotherapy is of particular interest. Therefore the present invention further provides a triazole derivative as defined in [3] for use in a method of treatment of cancer of the human or animal body by radiotherapy, wherein said triazole derivative comprises at least one drug moiety. The present invention further also provides use of a triazole derivative as defined in [3], in the manufacture of a medicament for use in the method of treatment of cancer of the human or animal body by radiotherapy, wherein said triazole derivative comprises at least one drug moiety. In these embodiments, X is preferably ¹³¹I or ²¹¹At.

The invention further provides a method of treatment, preferably of cancer by radiotherapy, of the human or animal body which comprises administering to a human or animal patient a triazole derivative as defined in [3], wherein said triazole derivative comprises at least one drug moiety.

Some of the embodiments of this invention are concerned with the fields of diagnosis and therapy. When a relevant diagnostic and/or therapeutic method is performed, the triazole derivative must first be administered to the human or animal body (the “patient”). It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, but an exemplary dosage would be 0.1-1000 mg per day.

The medical compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers, for example lactose, sugar, maize-starch, calcium phosphate, sorbitol or glycine; tabletting lubricant, for example magnesium stearate, talc, polyethylene glycol or silica; disintegrants, for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the medical compounds may be made up into a cream, lotion or ointment. Cream or ointment formulations which may be used for the drug are conventional formulations well known in the art, for example as described in standard textbooks of pharmaceutics such as the British Pharmacopoeia.

For topical application by inhalation, the medical compounds may be formulated for aerosol delivery for example, by pressure-driven jet atomizers or ultrasonic atomizers, or preferably by propellant-driven metered aerosols or propellant-free administration of micronized powders, for example, inhalation capsules or other “dry powder” delivery systems. Excipients, such as, for example, propellants (e.g. Frigen in the case of metered aerosols), surface-active substances, emulsifiers, stabilizers, preservatives, flavourings, and fillers (e.g. lactose in the case of powder inhalers) may be present in such inhaled formulations. For the purposes of inhalation, a large number of apparatuses are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described in European Patent Application EP 0 505 321).

For topical application to the eye, the medical compounds may be made up into a solution or suspension in a suitable sterile aqueous or non aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

The medical compounds of the invention may be used in conjunction with a number of known pharmaceutically active substances.

Still further, the present invention provides a process for producing the radioisotopically labelled alkyne represented by the formula (II′). In this embodiment, the group Y and preferred aspects thereof are as described above, while X is preferably selected from ¹⁸F, ¹²³I, ¹²⁴I, ¹²⁵I, ¹³¹I and ²¹¹At. More preferably X is selected from ¹²³I, ¹²⁴I, ¹²⁵I and ¹³¹I.

The inventors have found that the reaction between the terminal alkyne represented by the formula (II) and the radioisotopic halogen anion proceeds with high yield in the presence of Cu(II) ions and a base. Preferred reaction conditions are identical to those explained above in relation to production of the derivative represented by the formula (I), except that no azide represented by the formula (III) is used.

This process is a convenient means for introducing radiolabels and/or radiotherapeutic drug moieties into an alkyne. Such alkynes may in some circumstances be directly applicable for diagnostic and/or therapeutic purposes. Alternatively, they can be converted into a triazole derivative of formula (I) in a second step which comprises reacting the radioisotopically labelled alkyne represented by the formula (II′) with an azide represented by the formula (III), again in the presence of Cu(II) ions and a base. The preferred reaction conditions for this second step are also identical to those explained above in relation to production of the derivative represented by the formula (I). It will be appreciated that this second step can be carried out in the same reaction medium as that used to prepare the radioisotopically labelled alkyne represented by the formula (II′). Alternatively the radioisotopically labelled alkyne represented by the formula (II′) can be isolated and/or stored, ready for later derivitisation to produce the triazole derivative of formula (I).

### EXAMPLES

### Abbreviations

NIS N-iodosuccinimide
NBS N-bromosuccinimide
TSTU O-(N-succinimidyl)-1,1,3,3-tetramethyluronium tetrafluoroborate

### Example 1

In this Example a model trifunctionalised triazole incorporating radioiodine was constructed.

Benzyl azide (1) and N-benzyl-propargyl amide (2) were prepared according to method reported in Li et al., J. Org. Chem., 2008, 73: 3630-3633. ‘Click’ coupling of the above reagents under anhydrous conditions in the presence of triethylamine, excess CuI and NIS provided the iodinated 1,2,3-triazole 3 in quantitative yield within 30 min at room temperature (Scheme 1).

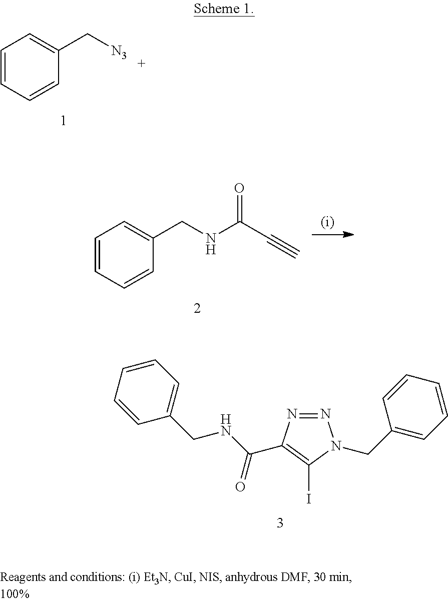

The procedure of Scheme 1 is not, however, suitable for production of radiolabelled analogues of compound 3. For example, it is important that only radioactive iodine is incorporated (ruling out the use of CuI or NIS) and since radioiodine is supplied in aqueous solutions the use of anhydrous reaction conditions is not appropriate. Moreover, under the literature conditions it would not be possible to obtain the trifunctionalised product when using a radioiodine reagent, since the concentration of the radiolabel is minute compared to that of the other reagents: thus the competing two-component reaction between the azide and the alkyne would be overwhelmingly dominant.

Reaction conditions were therefore investigated that involved use of aqueous solutions of n.c.a. (non-carried added) radioiodine (supplied as Na¹²⁵I, specific activity of 629 GBq/mg, 5 MBq used per reaction), technically undemanding chemistry (i.e. wet conditions and addition of all reagents as solutions) and mild conditions (room temperature, 15 min). The results of these studies are summarized in Table 1.

TABLE 1
Reaction conditions and yields for model compound [¹²⁵I]3; all reactions
were carried out in the presence of 2.2 equivalents of Et₃N to copper

| | 1^{st} Cu | 2^{nd} Cu | | | |
|---|---|---|---|---|---|
| | source | source | Solvent | Azide:alkyne | Yield of |
| Entry | (1 equiv.) | (1 equiv.) | (100 μl) | 1:1 | [¹²⁵I]3 |
| 1 | CuOAc | Cu(OAc)₂ | DMF | 10 μmol | 30% |
| 2 | CuCl | CuCl₂ | DMF | 1 μmol | 41% |
| 3 | CuCl₂ | none | DMSO | 1 μmol | 21% |
| 4 | CuCl₂ | none | AcCN | 1 μmol | 46% |

Radiochemical yields were measured using radio-HPLC (analytical, not isolated). The one-step labeling reaction preceded with comparable radiochemical yields to what is typically achieved when labeling aromatic groups with the ‘gold standard’ iododestannylation reaction. Only 1 μmol of each of the starting materials was required for the reaction to proceed, enabling purification of the radiolabelled product by HPLC using semi-preparative columns.

Notably, the best yield (entry 4) was obtained in the absence of any added Cu(I) ions, clearly demonstrating that the desired reaction proceeds efficiently in the presence of a source of Cu(II) ions.

### Example 2

In this Example a model labelling reagent incorporating radioiodine and a fluorescent group was constructed.

6,8-difluoro-7-hydroxy-coumarin 7 was used as the fluorescent reporter. The 4-chloromethyl-6,8-difluoro-7-hydroxy-coumarin (6) was prepared in 67% yield by a Pechmann condensation of 2,4-difluororesorcinol (4) and ethyl 4-chloroacetoacetate (5) (Scheme 2). Treatment with sodium azide provided the coumarin azide precursor 6 in 52% yield. To enable conjugation of the labelling reagent to biomolecules, pentafluorophenyl propinoicate 8 was used as the alkyne precursor.

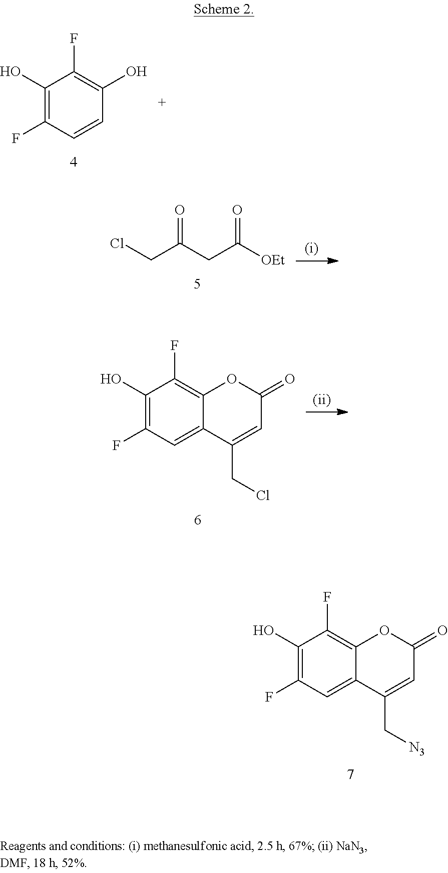

The three component one-pot “click” coupling between coumarin azide 7 and pentafluorophenyl propinoicate (8) in the presence of an electrophilic iodine source was investigated (Scheme 3). The initial attempt was based on the method reported by Wu (Y. M. Wu, J. Deng, Y. Li, Q. Y. Chen, Synthesis, 2005, 1314.) using ICI as iodination reagent and triethylamine as ligand and base. However, it was found that the coumarin azide 7 and the pentafluorophenyl ester 8 rapidly decomposed under these conditions.

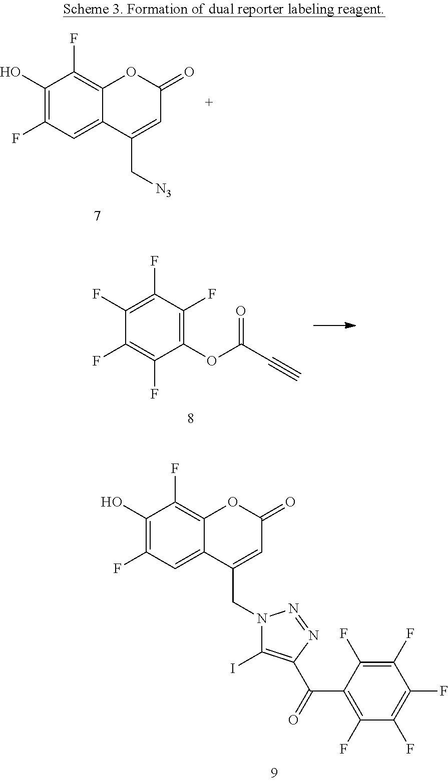

Fukase (K. Tanaka, C. Kageyama and K. Fukase, J. Med. Chem., 2007, 48, 6475) have reported that histidine derivatives are excellent ligands for formation of iodinated triazoles. Therefore, it was decided to employ the double N-protected N-Boc-L-His(Tos)-OH as the ligand in the presence of CuI and N-iodo succinimide. The reaction was completed in 30 min and gave the desired iodinated coumarin pentafluorophenyl ester as the single product with 100% conversion detected by LC-MS.

However, it was difficult to separate the product from the histidine ligand by column chromatography. An alternative route to simplify the purification process is to modify the histidine ligand so that it can be either removed by a column or a solvent wash. Two new histidine ligands were prepared and subsequently used for the “click reaction”. These new histidine ligands had the formula

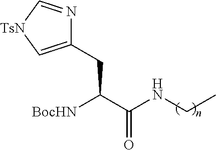

with n being 4 and 11, respectively.

It was found that both ligands have similar copper chelating capabilities as the N-Boc-L-His(Tos)-OH and gave 100% conversion according to LC-MS analysis. The C₁₂ ligand enabled simple separation from the iodinated product.

Following isolation of the coumarin labelling reagent 9 conditions for labelling with iodine-125 were investigated. The conditions used and the results are outlined in Table 2. All reactions were carried out in DMF at room temperature with 15 min reaction time.

TABLE 2
Reaction conditions and yields for model compound [¹²⁵I]9.

| | Catalyst | Oxidant | Azide:Alkyne | Yield of |
|---|---|---|---|---|
| Entry | (1 equiv.) | (1 equiv.) | 1:1 | [¹²⁵I]9 |
| 1 | CuI | none | 10 μmol | <5% |
| 2 | CuOAc | none | 10 μmol | 9% |
| 3 | CuCN | none | 10 μmol | 12% |
| 4 | CuOAc | H₂O₂ | 10 μmol | 19% |
| 5 | CuOAc | Cu(OAc)₂ | 10 μmol | 37% |
| 6 | Cu(OAc)₂ | none | 10 μmol | 39% |

### Example 3

In this Example a labelling reagent incorporating radioiodine, a fluorescent group and an antibody was constructed and used to image stained human colorecteral tumour tissues.

Preliminary imaging studies with an antibody labelled with the coumarin-based dual reporter reagent 9 indicated that the fluorescent group was prone to photobleaching. In order to improve the optical imaging properties of the iodinated triazole the coumarin group was replaced with Rhodamine B. Rhodamine is widely used for optical imaging and is commercially available at low cost (£21 for 100 g as of July 2010). The commercially available rhodamine B was converted to the corresponding acid chloride and reacted with N-methyl-propargylamine to provide the new alkyne precursor 12 (Scheme 4) in 72% yield.

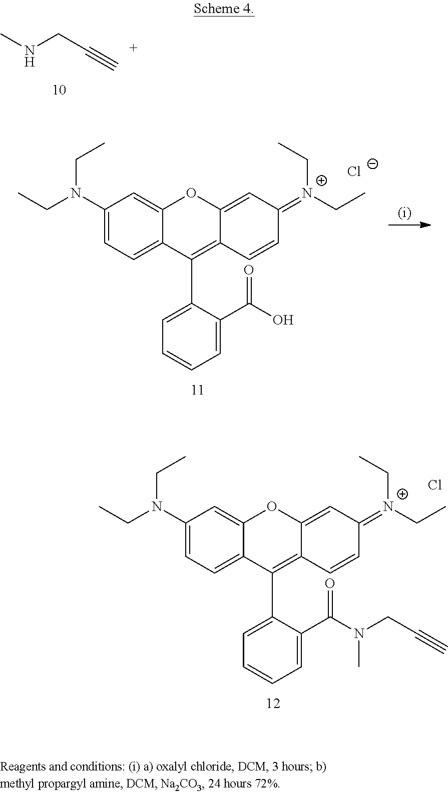

4-azidomethyl-N-succinimidyl benzoate (15) was prepared in two steps (Scheme 5). First, the 4-chloromethyl benzoic acid (13) was refluxed with sodium azide in ethanol for 18 hours to yield the corresponding azide 14 in 94% yield. This azide was subsequently coupled with TSTU in the presence of trimethyl amine to form the 4-azidomethyl-N-succinimidyl benzoate (15) in 69% yield.

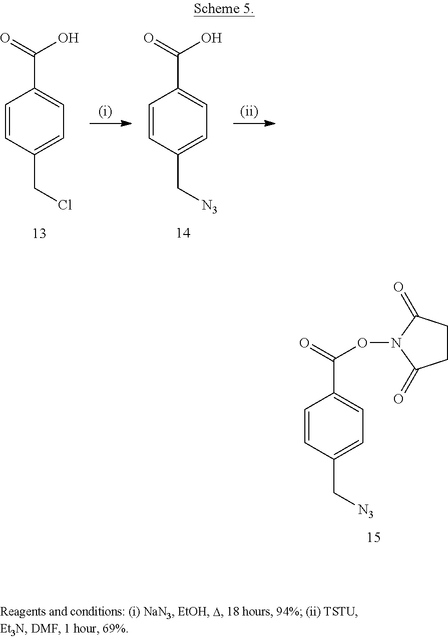 [SMILES: O=C(O)c1ccc(CCl)cc1 ; [N-]=[N+]=NCc1ccc(C(=O)O)cc1 ; [N-]=[N+]=NCc1ccc(C(=O)ON2C(=O)CCC2=O)cc1]

The rhodamine B methyl propargyl amide 12 and 4-azidomethyl-N-succinimidyl benzoate (15) were subjected to the standard “click” reaction previous described (Scheme 6). It was observed that the desired iodinated triazole can be obtained in 92% yield within 2 h reaction time using acetonitrile as the solvent.

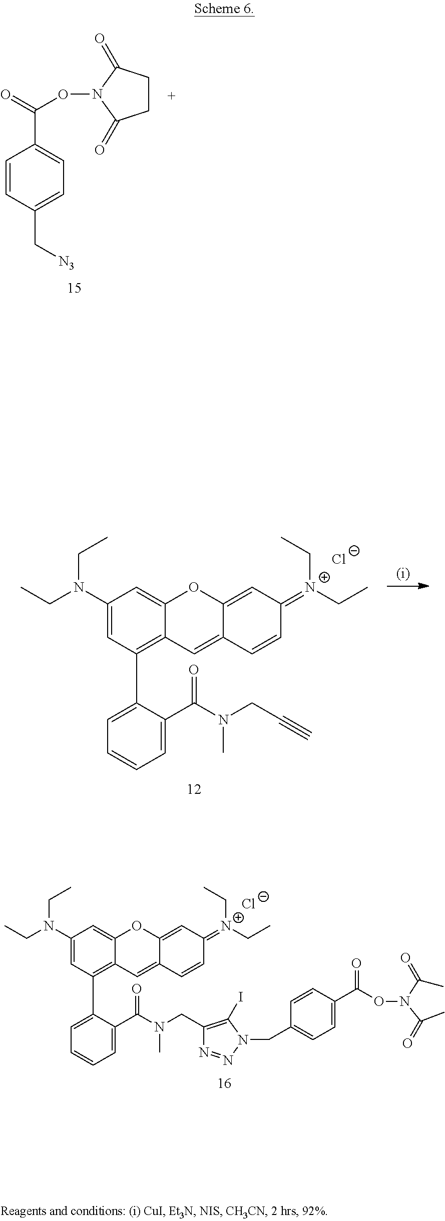

The reaction between rhodamine B methyl propargyl amide 12 and 4-azidomethyl-N-succinimidyl benzoate 15 in the presence of iodide-125 was initially carried out under the conditions described for the model system (scheme 7).

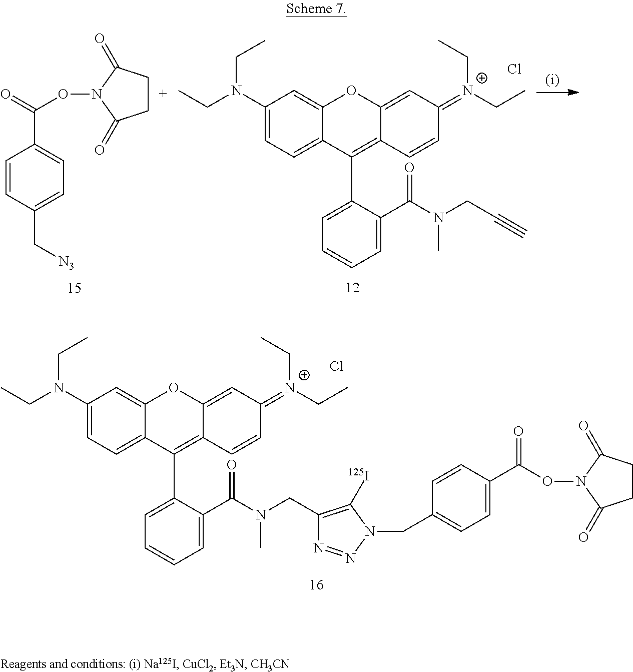

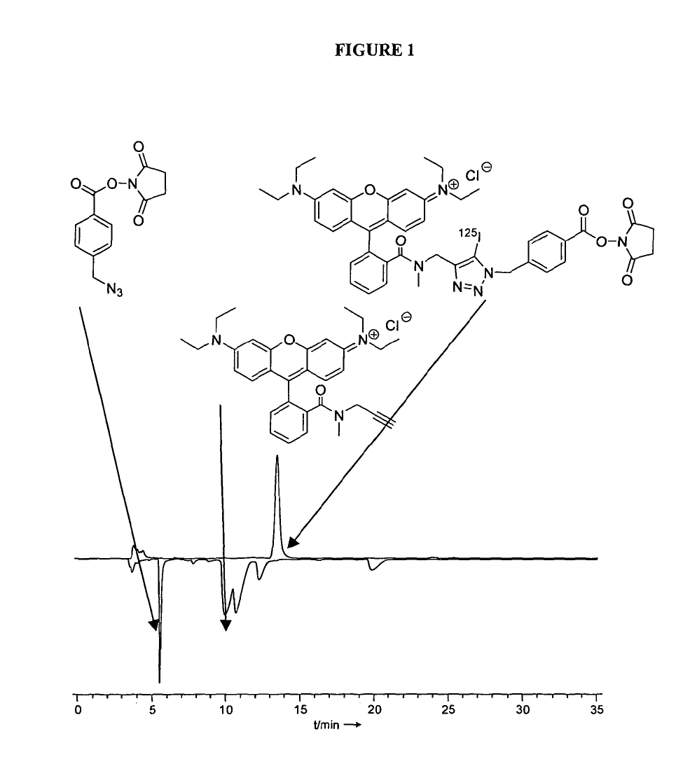

This afforded 25% analytical radiochemical yield of [¹²⁵I]16 in 15 min reaction time at room temperature. However, when the amount of Et₃N used was changed to 1.5 equivalents (relative to the amount of CuCl₂) and the CuCl₂, Et₃N and rhodamine B methyl propargyl amide were pre-mixed in the CH₃CN, the analytical radiochemical yield increased to 74% in 30 minutes and 83% in 90 minutes. High purity of rhodamine B ¹²⁵I triazole was obtained after reverse-phase HPLC purification as shown in FIG. 1.

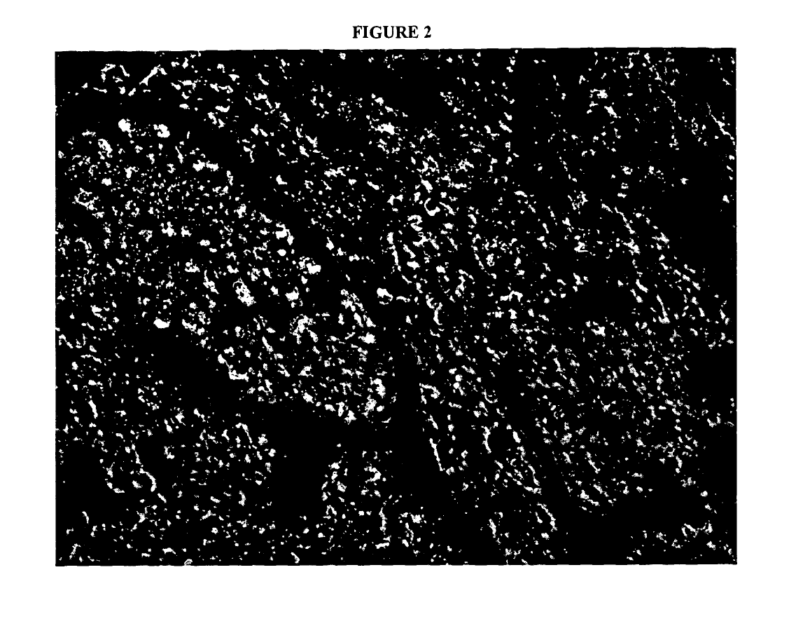

A monoclonal anti-CEA antibody (A5B7) widely used in radioimmunotherapy was selected for the labelling and fluorescent staining study of the new rhodamine B iodo triazole labelling group. It was found that either one or two labelling groups can be coupled onto this antibody in a pH 9.0, 1 M carbonate buffer at room temperature in one hour with 10 or 25 equivalences of the labelling group. These two samples were subsequently applied to the fluorescent staining study of two types of human colorecteral tumour tissues-SW1222 and LS1.74T with 5 μL/mL and 20 μL/mL of labelled A5B7. Both tumour tissues were successfully stained under both concentrations of the labelled A5B7 with either one or two rhodamine B iodo triazole labelling groups and high quality images of these stained tissues were obtained as shown in FIG. 2.

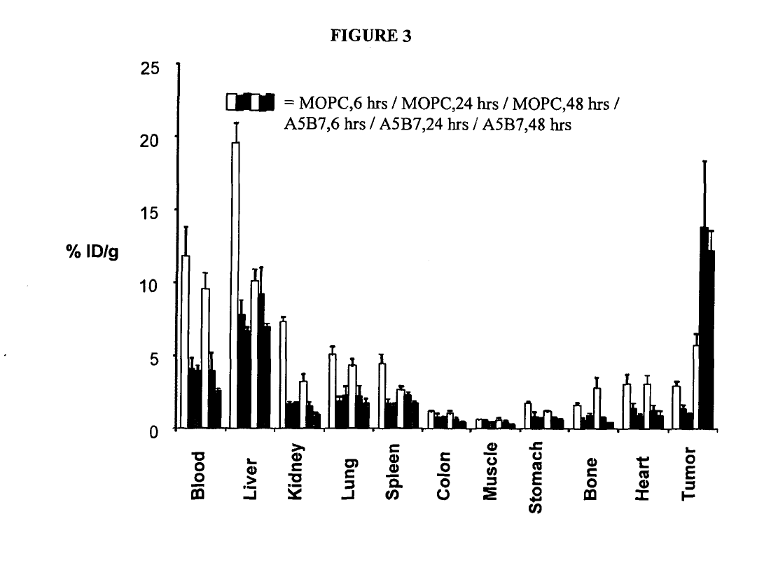

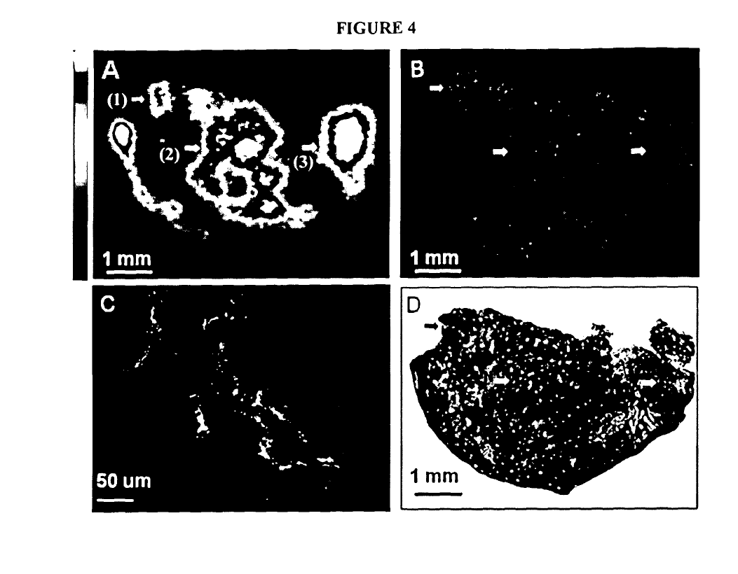

Dual antibody labeling was also achieved by incubating a solution of A5B7 with a mixture of the rhodamine B [¹²⁵I] iodo triazole (20 MBq) and non-radioactive labeling reagent (20 equiv.) at room temperature for 1 hour in 22% RCY with an average of 6-8 fluorescent groups per antibody. The dual labeled A5B7 was subsequently evaluated in mice bearing human colorectal xenografts. Its overall organ distribution shown in FIG. 3 was similar to that of the antibody when labeled with ¹²⁵I alone using the Chloramine-T method. The tumor uptake of dual labeled A5B7 24 h and 48 h after injection was 13.9±4.5% ID/g and 12.3±1.3% ID/g, respectively. Good clearance was observed for all organs apart from the liver, which had an increased uptake (6.99±0.16% ID/g at 48 h). Interestingly, the increased liver uptake was accompanied by a more rapid blood clearance, resulting in tumor-to-blood ratios of 3.5:1 and 4.8:1 at 24 h and 48 h post-injection, respectively. Frozen tumor sections were used to visualize antibody localization across the whole tumor 6 h after injection of 50 μg of dual labeled A5B7. The distribution of radioactivity (FIG. 4A) was in good agreement with the fluorescence signal (FIG. 4B), and demonstrated antibody uptake across the viable areas of the tumor (FIG. 4D). High magnification fluorescence imaging (FIG. 4C) showed that the antibody was associated mainly with tumor cells around perfused blood vessels, but also had started to diffuse away from vessels into the tumor mass at this early time post-injection.

It was also found that the RCY of A5B7 labelling was increased to 46%±5 (n=3) when only the rhodamine B [¹²⁵I]iodo triazole was used for the conjugation reaction (non-carrier-added).

### Example 4

To further demonstrate the application of this three component “click” chemistry, a dansyl derived dye modified with a thiol selective maleimide group was prepared for site specific labelling of proteins and cells.

The two “click” components were prepared as following. Propargylamine and maleic anhydride was stirred in acetic acid for 18 hours to form the carboxylic acid intermediate which was suspended in acetic anhydride in the presence of sodium acetate at 65° C. for 2 hours. The N-propargyl maleimide was obtained in 28% yield.

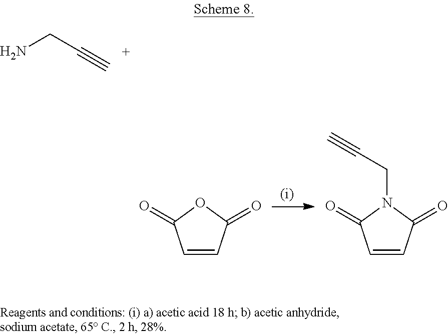

The dansyl ethyl azide was obtained in 69% yield by reacting dansyl chloride with 2-bromoethylamine hydrobromide in the presence of triethylamine and then refluxing the intermediate with sodium azide for 18 hours.

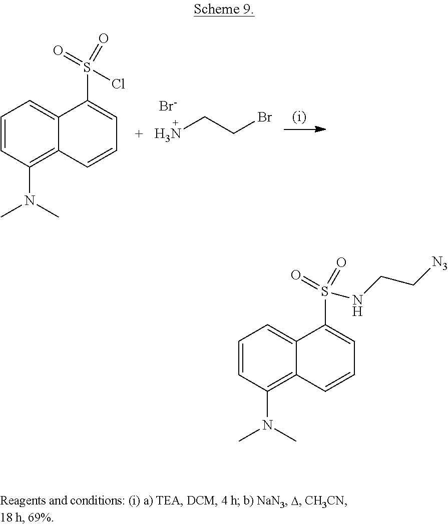

The dansyl ethyl azide and the propagyl maleimide were coupled in the presence of CuI and NIS as described above (80C.° for 18 h) to give the resulting non-radioactive iodinated triazole in 55% yield.

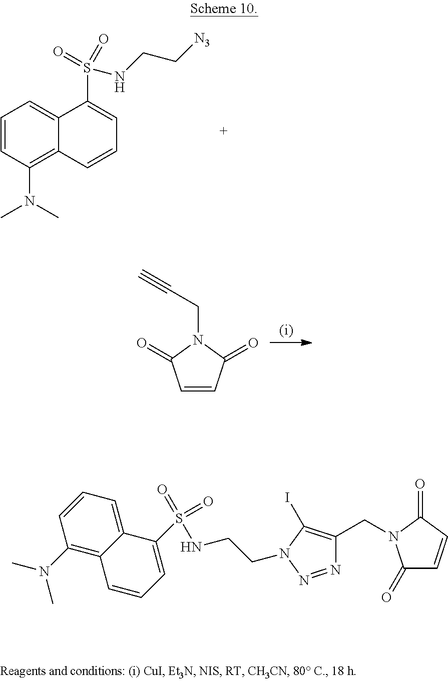

The dansyl ethyl azide and the propagyl maleimide were used to obtained a dual labelling reagent by submitting the reagents to the radiochemical labelling conditions described in previous examples. With the exception of heating the reaction mixture at 60° C. for 90 min. This resulted in formation of the dual labelling reagent in 91% analytical radiochemical yield.

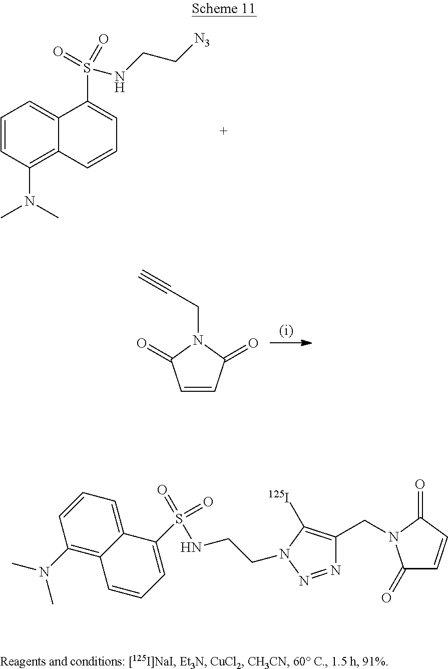

### Experimental Details for the Examples

### N-benzyl-propargyl amide

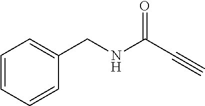

To a solution of propiolic acid (0.64 mL, 10.0 mmol) and DCC (2.50 g, 12.0 mmol) in DCM (60 mL) were added benzyl amine (1.09 g, 10.0 mmol). The reaction was stirred at room temperature for 24 hours. The mixture was filtered through a Buchner funnel. The solvent was removed and the crude material was purified by column chromatography on silica, eluting with DCM and MeOH (60:1) to yield the title compound as colourless powder (0.63 g, 40%); ¹H NMR (300 MHz; CDCl₃) δ 7.36-7.28 (5H, m, Ph), 6.31 (1H, broad s, NH), 4.47 (2H, d, J=11.74, CH₂), 2.79 (1H, s, alkyne); ¹³C NMR (75 MHz; CDCl₃) δ 152.0, 137.0, 128.9, 128.0, 127.2, 79.8, 73.6, 43.9; m/z (E.I.), 160 (MH⁺, 100%), 159 (M⁺, 7%), and 91 (20%); HRMS for C₁₀H₁₀NO requires 160.0762 found 160.0768.

### Benzyl azide

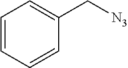

To a solution of benzyl bromide (1.22 g, 10.0 mmol) in dimethyl sulfoxide (15 mL) was added sodium azide (2.00 g, 30.0 mmol) and sodium iodide (4.50 g, 30.0 mmol). The suspension was warmed at 50° C. for 18 hours. The mixture was cold to room temperature and quenched with water (50 mL) and the mixture was extracted with diethyl ether (3×50 mL). The organic phase was washed with brine and dried over MgSO₄. The solvent was removed in vacuum to yield the title compound as colourless oil (1.33 g, 100%); ¹H NMR (300 MHz; CDCl₃) δ 7.40-7.31 (5H, m, Ph), 4.34 (2H, s, CH₂).

### N-Benzyl-1-benzyl-5-iodo-1,2,3-thiazole-4-carboxamide

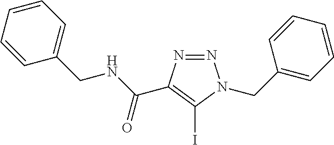 [SMILES: O=C(NCc1ccccc1)c1nnn(Cc2ccccc2)c1I]

To a suspension of copper (I) iodide (95 mg, 0.5 mmol) and triethyl amine (0.09 mL, 0.6 mmol, 1.2 equiv.) in dry DMF (1.5 mL) was added benzyl azide (67 mg, 0.50 mmol), N-benzyl-propargyl amide (80 mg, 0.50 mmol) and N-iodosuccinamide (115 mg, 0.50 mmol) under Ar. The mixture was stirred for 30 min. The reaction was quenched by addition of water (15 mL). The precipitate was collected on a Buchner funnel and purified by flash column chromatography on silica, eluting with DCM-methanol (12:1), to furnish the title compound as yellow powder (208 mg, 100%); ¹H NMR (300 MHz; CDCl₃) δ 9.14 (2H, s, CH₂), 7.30-7.18 (10H, m, Ph), 5.70 (2H, s, NCH₂), 4.41 (2H, s, NHCH₂); ¹³C NMR (75 MHz; CDCl₃) δ 159.2, 139.5, 135.0, 128.7, 128.2, 127.3, 126.7, 53.2, 41.9; m/z (E.I.), 419 (MH⁺, 100%), 418 (M⁺, 50%), 391 (51%), 382 (18%), and 264 (10%); FIRMS for C₁₇H₁₆IN₄O requires 419.0369 found 419.0361.

### 4-chloromethyl-6,8-difluoro-7-hydroxy-coumarin

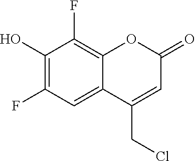 [SMILES: O=c1cc(CCl)c2cc(F)c(O)c(F)c2o1]

A solution of 2,4-difluororesorcinol (2.00 g, 13.7 mmol) and ethyl 4-chloroacetoacetate (1.8 mL, 17.5 mmol) in methanesulfonic acid (15 mL) was stirred at room temperature for 2.5 hours, then quenched with water (150 mL). The resulting precipitate was collected on a Buchner funnel and dried to give the title compound as a colourless powder (2.25 g, 67%); ¹H NMR (300 MHz; d₆-DMSO) δ 7.53 (1H, dd, J=2.17, J′=11.44, FCCH), 6.55 (s, 1H, CH₂C═CH), 4.92 (s, 2H, CH₂); ¹³C NMR (d₆-DMSO) δ 158.6, 150.4, 146.7, 139.8, 137.8, 113.2, 108.2, 106.3, 106.0, 41.0; m/z (E.I.), 247 (M⁺, (³⁷Cl) 37%), 245 (M⁺, (³⁵Cl) (100%), 209 (60%), 181 (96%), and 153 (50%); HRMS for C₁₀H₄ClF₂O₃ requires 244.9817 found 244.9813.

### 4-Azidomethyl-6,8-difluoro-7-hydroxy-coumarin

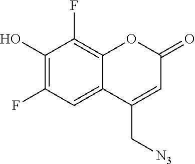 [SMILES: [N-]=[N+]=NCc1cc(=O)oc2c(F)c(O)c(F)cc12]

To a solution of 4-chloromethyl-6,8-difluoro-7-hydroxycoumarin (131 mg, 0.53 mmol) in DMF (5 mL) was added NaN₃ (104 mg, 1.63 mmol, 3.0 equiv.). The resulting mixture was stirred at room temperature for 18 hours before quenched with water (10 mL). The precipitate was collected on a Buchner funnel and dried to give the title compound as a pale yellow powder (70 mg, 52%); ¹H NMR (300 MHz; d₆-DMSO) δ 7.43 (1H, dd, J=2.16, J′=11.40, FCCH), 6.41 (1H, s, CH₂C═CH), 4.76 (2H, s, CH₂); ¹³C NMR (75 MHz; d₆-DMSO) δ 158.5, 149.7, 146.8, 139.6, 137.80, 111.4, 108.2, 106.0, 105.7, 49.5; m/z (E.I.), 252 (M⁺, 28%), 224 (68%), 197 (66%), and 169 (100%); HRMS for C₁₀H₄F₂N₃O₃ requires 252.0221 found 252.0229.

### Pentafluorophenyl propinoicate

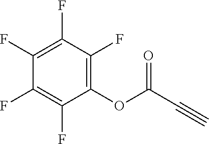 [SMILES: C#CC(=O)Oc1c(F)c(F)c(F)c(F)c1F]

To a solution of propiolic acid (1.00 g, 14.3 mmol) and DCC (3.25 g, 16.0 mmol) in DCM (60 mL) were added pentafluorophenol (2.10 g, 11.4 mmol). The reaction was stirred at room temperature for 48 hours. The mixture was filtered through a Buchner funnel. The solvent was removed and the crude material was purified by column chromatography on silica, eluting with petrol and ether (10:1) and then recrystallised from petrol to yield the title compound as colourless needles (1.75 g, 52%); ¹H NMR (300 MHz; CDCl₃) δ 3.26 (1H, s, alkyne); ¹³C NMR (75 MHz; CDCl₃) δ 147.9, 79.7, 72.1; m/z (E.I.), 236 (MH⁺, 5%), 185 (5%), 134 (8%), and 85 (19%); HRMS for C₉H₂F₅O₂ requires 236.9975 found 236.9986.

### Pentafluorophenyl 1-[4-methyl-6,8-difluoro-7-hydroxy]-coumarin-5-iodo-1,2,3-thiazole-4-carboxylate

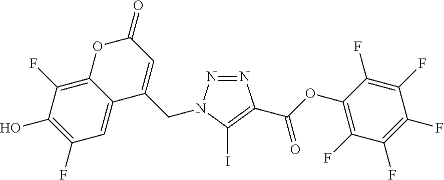 [SMILES: O=C(Oc1c(F)c(F)c(F)c(F)c1F)c1nnn(Cc2cc(=O)oc3c(F)c(O)c(F)cc23)c1I]

To a suspension of copper (I) iodide (95 mg, 0.5 mmol) and N-dodecyl-Nₐ-Boc-N₍ᵢₘ₎-tosyl-L-histidinamide (290 mg, 0.5 mmol) in dry DMF (1.5 mL) was added 4-azidomethyl-6,8-difluoro-7-hydroxy-coumarin (125 mg, 0.50 mmol), pentafluorophenyl propinoicate (120 mg, 0.50 mmol) and N-iodosuccinamide (115 mg, 0.50 mmol) under argon. The solution was stirred for 30 minutes. The reaction was quenched by addition of water (5 mL) The precipitate was collected on a Buchner funnel and purified by flash column chromatography on silica, eluting with DCM-methanol (12:1), to furnish the title compound as yellow powder (151 mg, 49%); ¹H NMR (300 MHz; CDCl₃) δ 7.89 (1H, s, CFCH), 7.55 (1H, Broad s, OH), 6.10 (2H, s, CH₂), 5.65 (1H, s, COCH); ¹³C NMR (75 MHz; CDCl₃) δ m/z (E.I.), 616 (MH⁺, 100%), 490 (18%), 220 (22%), and 185 (33%); HRMS for C₁₉H₅F₇N₃O₅ requires 615.9240 found 615.9248.

### N-Boc-L-histidine (Tos)-N

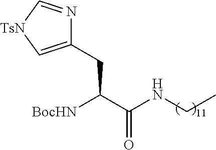

To a suspension of N-Boc-L-His(Tos)-OH (1.23 g, 3.0 mmol) and DCC (681 mg, 3.0 mmol) in DCM (30 mL) were added dodecylamine (612 mg, 3.3 mmol). The reaction was stirred at room temperature for 24 hours. The mixture was filtered through a Buchner funnel. The solvent was removed and the crude material was purified by column chromatography on silica, eluting with DCM and methanol (40:1) and to yield the title compound as white powder (380 mg, 29%); ¹H NMR (300 MHz; CDCl₃) δ 7.92 (1H, s, NCHN), 7.79 (2H, d, J=8.2, SCCH), 7.34 (2H, d, J=8.2, SCCHCH), 7.08 (1H, s, CHNS), 6.75 (1H, s, CH₂NH), 5.83 (1H, d, J=7.3, CHNH), 4.60 (1H, broad s, CHNH), 3.11-3.07 (2H, m, CH₂NH), 2.43 (3H, s, CH₃), 1.71-1.66 (2H, m, NHCHCH₂), 1.39 (9H, s, CH₃×3), 1.35-1.02 (20H, m, CH₂×20), 0.93-0.84 (3H, m, CH₂CH₃); ¹³C NMR (75 MHz; CDCl₃) δ 170.9, 155.2, 146.3, 140.8, 136.1, 134.8, 130.4, 127.4, 114.9, 49.0, 80.1, 68.2, 39.4, 34.0, 31.9, 30.3, 29.6, 29.5, 29.3, 29.2, 28.3, 26.8, 22.7, 21.7, 14.1; m/z (E.I.), 599 (MNa⁺, 40%), 556 (36%), 187 (40%), and 186 (100%); HRMS for C₃₀H₄₈N₄O₅NaS requires 599.3243 found 599.3267.

### 4-azidomethyl benzoic acid

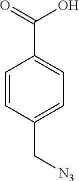 [SMILES: [N-]=[N+]=NCc1ccc(C(=O)O)cc1]

To a solution of 4-chloromethyl benzoic acid (1.71 g, 10.0 mmol) in absolute ethanol (50 mL) was added sodium azide (1.70 g, 26.0 mmol). The suspension was refluxed for 18 hours. The mixture was cold to room temperature and quenched with water (100 mL). The solvents were partially removed in vacuum and cooled to 0° C. The solution was acidified by addition of concentrated HCl to pH 2. The precipitate was collected through a Buchner funnel to yield the title compound as white powder (1.67 g, 94%); ¹H NMR (300 MHz; d₆-DMSO) δ 7.95 (2H, d, J=8.23, CHCCO), 7.50 (2H, d, J=8.23, CHCCH₂), 4.55 (s, 2H, CH₂); ¹³C NMR (75 MHz; d₆-DMSO) δ 166.9, 140.5, 130.4, 129.6, 128.3, 53.0; m/z (E.I.), 178 (M⁺, 178, 100%), 160 (20%), 150 (17%), and 135 (60%); HRMS for C₈H₈N₃O₂ requires 178.0616 found 178.0623.

### 4-azidomethyl-N-succinimidyl benzoate

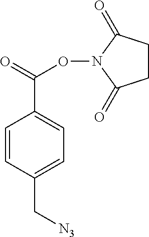 [SMILES: [N-]=[N+]=NCc1ccc(C(=O)ON2C(=O)CCC2=O)cc1]

To a solution of 4-azidomethyl benzoic acid (885 mg, 5.0 mmol) and triethyl amine (0.71 mL, 5.0 mmol) in DMF (30 mL) was added TSTU (1.66 g, 5.5 mmol). The resulting solution was stirred at room temperature for 1 hour before cooling to 0° C. The reaction was quenched with 2 M HCl (50 mL) and water (25 mL). The precipitate was collected through a Buchner funnel to yield the title compound as white needles (942 mg, 69%); ¹H NMR (300 MHz; CDCl₃) δ 8.15 (2H, d, J=8.29, CHCCO), 7.54 (2H, d, J=8.29, CHCCH₂), 4.47 (s, 2H, N₃CH₂), 2.91 (s, 2H, CH₂); ¹³C NMR (75 MHz; CDCl₃) δ 169.2, 161.5, 142.8, 131.1, 128.3, 124.9, 54.1, 25.7; m/z (E.I.), 275 (M⁺, 275, 10%), 247 (17%) and 160 (100%); HRMS for C₁₂H₁₁N₄O₄ requires 275.0780 found 275.0785.

### N-methyl N-propargyl rhodamine B carboxamide

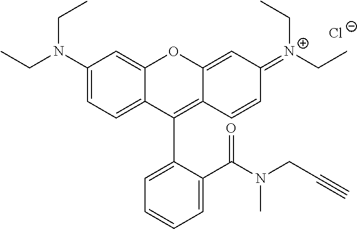 [SMILES: C#CCN(C)C(=O)c1ccccc1-c1c2ccc(=[N+](CC)CC)cc-2oc2cc(N(CC)CC)ccc12 ; [Cl-]]

To a solution of Rhodamine B (479 mg, 1.0 mmol) in DCM (30 mL) was added oxalyl chloride (2.50 mL, 30.0 mmol) and the resulting solution was stirred for 1 hour at room temperature. Solvent and excess of oxalyl chloride were removed under reduced pressure. The resulting purple solid was dissolved in DCM (30 mL) to which Na₂CO₃ (anhydr.) (530 mg, 5.0 mmol) and N-methylpropargylamine (0.44 mL, 5.0 mmol) were added. The reaction was stirred for 18 hours at room temperature. After filtration, solvent and excess of N-methylpropargylamine were removed under reduced pressure to give a purple residual solid. The crude material was purified by flash column chromatography, eluting with DCM and methanol (15:1) to yield the title compound as purple powder (383 mg, 72% yield); ¹H NMR (400 MHz, DMSO-d₆, 80° C.) δ 7.77-7.75 (2H, m, Ar), 7.67-7.66 (1H, m, Ar), 7.55-7.53 (1H, m, Ar), 7.20 (0.5H, s, Ar), 7.17 (1.5H, s, Ar), 7.11 (1.5H, d, J=2.48, Ar), 7.09 (0.5H, d, J=2.48, Ar), 6.93 (2H, d, J=2.48, Ar), 4.01 (2H, s, CH₃NCH₂), 3.68 (8H, q, J=7.08, CH₃CH₂N×4), 2.85 (3H, s, CH₃N), 2.50 (1H, s, CHCCH₂), 1.26 (12H, t, J=7.08, CH₃CH₂N×4); ¹³C NMR (150 MHz, CDCl₃) δ 168.1, 157.7, 155.3, 135.3, 134.8, 131.8, 130.9, 130.5, 130.4, 130.2, 130.0, 129.9, 129.6, 127.5, 127.4, 114.2, 114.0, 113.5, 96.0, 81.1, 74.1, 71.6, 46.1, 41.3, 36.6, 35.0, 31.4, 12.6; mp: 77-78° C.; HRMS (FAB, m/z): cation C₃₂H₃₆N₃O₂ (M⁺) calc. 494.2807 found 494.2809.

### N-methyl N-[5-iodo-1-(4-methyl-N-succinimidyl benzoate)-4-methyl-1,2,3-thiazole]rhodamine B carboxamide

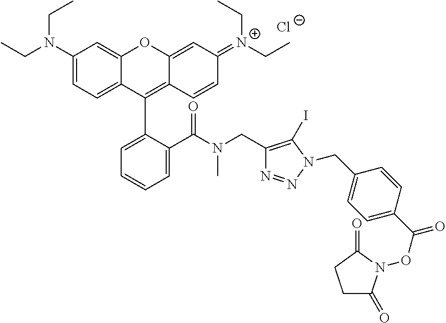 [SMILES: CCN(CC)c1ccc2c(-c3ccccc3C(=O)N(C)Cc3nnn(Cc4ccc(C(=O)ON5C(=O)CCC5=O)cc4)c3I)c3ccc(=[N+](CC)CC)cc-3oc2c1 ; [Cl-]]

To a solution of copper (I) iodide (38 mg, 0.20 mmol) and triethylamine (21 mg, 0.20 mmol) in dry acetonitrile (2 mL) were added the Rhodamine B alkyne 1 (106 mg, 0.20 mmol), 4-azidomethyl-N-succinimidyl benzoate (54 mg, 0.20 mmol) and N-iodosuccinimide (51 mg, 0.22 mmol, 1.1 equiv.) and the resulting mixture was stirred for 2 hours at room temperature under an atmosphere of argon. The solvent was removed under reduced pressure and the resulting solid was suspended in CHCl₃ (˜2 mL). The suspension was filtered through a short pad of Celite® and ethyl acetate (˜10 mL) was added to the filtrate. The resulting solid precipitate was collected by filtration and washed with ethyl acetate to yield the title compound as a purple powder (170 mg, 92% yield). If higher purity of the product was required, the crude material was purified by flash column chromatography on silica, eluting with DCM and methanol (20:1); ¹H NMR (400 MHz, DMSO-d₆, 80° C.) δ 8.10-8.08 (2H, m, Ar), 7.74-7.71 (3H, m, Ar), 7.52-7.50 (1H, m, Ar), 7.36-7.34 (2H, m, Ar), 7.20-7.17 (2H, m, Ar), 7.07-7.04 (2H, m, Ar), 6.91 (2H, d, J=2.00, Ar), 5.73 (2H, s, NNCH₂), 4.40 (2H, s, CH₃NCH₂), 3.66 (8H, q, J=7.04, CH₃CH₂N×4), 2.90 (4H, s, COCH₂×2), 2.80 (3H, s, CH₃N), 1.25 (12H, t, J=7.04, CH₃CH₂N×4); ¹³C NMR (150 MHz, DMSO-d₆) δ 179.5, 170.4, 167.3, 161.4, 157.2, 155.1, 147.3, 143.2, 135.9, 132.0, 131.7, 130.6, 130.4, 130.3, 129.8, 129.5, 128.2, 127.9, 127.1, 124.0, 113.9, 113.1, 95.8, 84.7, 59.8, 52.7, 45.4, 40.1, 36.9, 29.6, 25.6, 20.8, 14.1, 12.4; mp: 95-97° C.; HRMS (FAB, m/z): cation C₄₅H₄₅IN₆O₆ (M⁺) calc. 894.2602 found 894.2624.

### Radio Synthesis of RhB¹²⁵I Triazole N-Succinimide Ester:

A solution of copper (II) chloride (134 μg, 1.0 μmol) and triethylamine (151 μg, 1.5 μmol) in acetonitrile (40 μL) was added to the Rhodamine B alkyne (530 μg, 1.0 μmol). After 5 min the resulting solution was added to a mixture of 4-azidomethyl-N-succinimidyl benzoate (275 μg, 1.0 μmol) in acetonitrile (20 μL) and [¹²⁵I]NaI (20-40 MBq) in water (6.0 μL). After 90 min the reaction mixture was diluted with water and acetonitrile (10:1, 1.0 mL) and the resulting solution was purified by HPLC using a ZORBAX column (300SB-C18, 9.4×250 mm, 5 μm) with the following eluent: water (0.1% formic acid) as solvent A and methanol (0.1% formic acid) as solvent B, going from 60% of B to 70% of B in 30 min and going back to 60% of B in 2 min and remaining at 60% of B for an additional 3 min with a flow rate of 3 mL/min. The retention time of the title compound was 13.40 min. The labeled compound co-eluted with the non-radioactive reference compound. For antibody labeling the fraction that contained [¹²⁵I]3 was diluted with water (12 mL), the solution was passed through a Sep-Pak C18 light cartridge (Waters), the cartridge was washed with water (5 mL) and the radioactive product was eluted with acetonitrile/ethanol (1:1, 1 mL). The solvents were removed by a stream of nitrogen prior to bioconjugation.

### Non-Radioactive Antibody Conjugation:

A5B7 Antibody Labelling with RhB Iodinated Triazole N-Succinimide Ester:

A5B7 antibody (100 μL, 3.9 μg/μL) solution was justified to pH 8.8 by addition of sodium carbonate buffer (100 μL, 1 M, pH 9.0) to which RhB iodinated triazole N-succinimide ester (10 uL, 6.0 μg/μL, 25 equiv.) in DMF was added. The solution was incubated at room temperature for one hour. The solution volume was justified to 0.5 mL by addition of phosphate buffer (0.3 mL, 0.1 M, pH 7.0) and then loaded on a PD mini trap G-25 seize exclusion column. After the solution entered the column bed, around 80% the labelled antibody was eluted out of the column by addition of phosphate buffer (0.8 mL, 0.1 M, pH 7.0).

### Dual Labelling of Antibody Using Carrier-Added Conditions:

Antibody labelling with [¹²⁵I] and cold RhB iodinated triazole N-succinimide ester: a solution of non-radioactive labelling reagent 16 (62 μg, 20 equiv.) in DMF (13 μL) was added to [¹²⁵I]16 (20 MBq) followed by a solution of A5B7 (507 μg) in phosphate buffer (130 μL, pH 7) and sodium carbonate buffer (130 μL, 1.0 M, pH 9.0). The reaction mixture was incubated at room temperature for one hour and purified using a PD MiniTrap G-25 column (GE Healthcare) following the manufacturer's instructions. The dual labeled [¹²⁵I]16-A5B7 (4.74 MBq, 6-8 fluorescent groups per antibody) was collected in a total volume of 0.8 mL. The recovery efficiency of the purification step was determined to be 80±7% (n=3) in separate experiments. Under the conditions used the dual labeling reagent [¹²⁵I]16 was completely retained on the size exclusion column. The number of fluorescent groups incorporated was calculated by taking into account the specific activity of the dual labeling reagent [¹²⁵I]16, correcting the RCY for residual activity in the reaction vial, and loss of the antibody on the size exclusion column.

### Dual Labelling of Antibody Using Non-Carrier-Added Conditions:

Antibody Labelling with [¹²⁵I]RhB Iodinated Triazole N-Succinimide Ester:

Sodium carbonate buffer (130 μL, 1.0 M, pH 9.0) was added to A5B7 (507 μg, 3.9 mg/mL) in phosphate buffer (130 μL, pH 7) followed by addition of a solution of RhB125I in DMF (13 μL). The reaction mixture was incubated at room temperature for one hour and purified using a PD MiniTrap G-25 column (GE Healthcare) following the manufacturer's instructions. The dual labeled [125I]16-A5B7 was collected in a total volume of 0.8 mL with 46% (n=3) isolated radiochemical yield. The recovery efficiency of the purification step was determined to be 80±7% (n=3) in separate experiments. Under the conditions used the dual labeling reagent [¹²⁵I]16 was completely retained on the size exclusion column.

### N-Propagylmaleimide

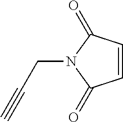

A solution of maleic anhydride (2.5 g, 25.5 mmol) and propargylamine (1.75 mL, 25.5 mmol) in glacial acetic acid (50 mL) was stirred in the dark overnight. The reaction mixture was concentrated to dryness. The crude material (1.49 g, 9.70 mmol) was suspended in acetic anhydride (7 mL) and sodium acetate (437 mg, 5.33 mmol) was added. The resulting suspension was stirred at 65° C. for 2 h, cooled down to room temperature, and then poured into ice-cold water (75 mL). The aqueous solution was extracted three times with diethyl ether. The combined organic layers were dried over MgSO₄, filtered, and concentrated. the crude material was purified by flash column chromatography on silica, eluting with hexane and EtOAc (2:1) provided the title compound (484 mg, 28%) as an off-white solid; mp: 52-53° C.; ¹H NMR (300 MHz, CDCl₃) δ 6.77 (2H, s, CH═CH), 4.28 (2H, s, CH₂), 2.21 (1H, s, CH); ¹³C NMR (75 MHz, CDCl₃) δ 169.3, 134.5, 77.5, 71.6, 26.8; m/z (E.I.), 135 (M⁺, 80%), and 107 (100%); HRMS for C₇H₅NO₂ requires 135.03148 found 135.03176.

### Dansyl ethyl azide

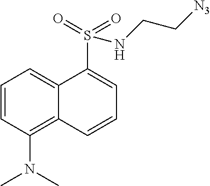 [SMILES: CN(C)c1cccc2c(S(=O)(=O)NCCN=[N+]=[N-])cccc12]

Dansyl chloride (2.7 g, 10 mmol) and bromoethylamine hydrobromide (2.05 g, 10 mmol) was stirred in CH₂Cl₂ (50 mL) for 4 hours in the presence of Et₃N (2.80 mL, 20 mmol). The solvent was evaporated and the residue was dissolved in MeCN (50 mL). NaN₃ (1.6 g, 25 mmol) was added and the mixture was refluxed overnight. After cooling to r.t. the solvent was removed and the product was purified on silica using petrol and diethyl ether (1:1) as eluent to afford the product as a greenish yellow oil (2.20 g, 69%); ¹H NMR (300 MHz, CDCl₃) 8.56 (1H, d, J=8.8 Hz, CSCH), 8.24-8.28 (2H, m, CH), 7.59 (1H, dd, J=7.3 Hz, J=8.8 Hz), 7.52 (1H, dd, J=7.3 Hz, J=8.8 Hz, CH), 7.20 (1H, d, J=7.3 Hz, CH), 5.08 (1H, t, J=5.8 Hz, NH), 3.27 (2H, t, J=5.8 Hz, CH₂N₃), 3.01-3.07 (2H, m, CH₂NH), 2.89 (6H, s, CH₃×2)¹³C NMR (75 MHz, CDCl₃) 171.1, 151.9, 144.4, 130.5, 129.7, 129.3, 128.5, 123.0, 118.4, 115.2, 50.9, 45.4, 42.4; m/z (E.I.), 135 (M⁺, 80%), and 107 (100%); HRMS for C₁₄H₁₈N₅O₂S requires 320.1181 found 320.1187.

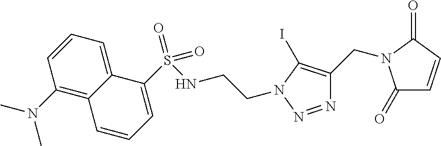 [SMILES: CN(C)c1cccc2c(S(=O)(=O)NCCn3nnc(CN4C(=O)C=CC4=O)c3I)cccc12]

To a solution of copper (I) iodide (95 mg, 0.50 mmol) and triethylamine (50 mg, 0.50 mmol) in dry acetonitrile (2 mL) were added dansyl ethyl azide (160 mg, 0.50 mmol), N-propagyl maleimide (68 mg, 0.50 mmol) and N-iodosuccinimide (125 mg, 0.55 mmol, 1.1 equiv.) and the resulting mixture was stirred for 2 hours at room temperature under an atmosphere of argon. The solvent was removed under reduced pressure and the resulting solid was suspended in DCM (5 mL). The suspension was filtered and the organic solution was washed with water (10×3 mL) and dried over MgSO₄. After filtration the solvent was removed under reduced pressure and the crude material was purified by flash column chromatography on silica, eluting with DCM and methanol (75:1); ¹H NMR (300 MHz, CDCl₃) δ 8.53 (1H, d, J=8.52, Ar), 8.23 (1H, J=6.13, Ar), 8.10 (1H, J=8.65, Ar), 7.54-7.48 (2H, m, Ar), 7.17 (1H, J=7.40, Ar), 6.74 (2H, s, CH═CH), 5.58 (1H, t, J=6.47, NH), 4.65 (2H, s, NHCH₂), 4.20 (2H, t, J=7.11, N₃CH₂), 3.50 (2H, q, J=6.26, CH₂NH), 2.98 (6H, s, CH₃×2); ¹³C NMR (75 MHz, CDCl₃) δ 169.9, 152.0, 146.2, 134.4, 130.8, 129.9, 129.6, 129.3, 128.7, 123.2, 118.5, 115.5, 79.3, 50.5, 45.4, 41.8, 33.3; m.p. 73-74° C.; HRMS (E.I., m/z): (M⁺) C₂₁H₂₂IN₆O₄S calc. 581.0468 found 581.0494.

### Radio Synthesis of [¹²⁵I] Dansyl Triazole Maleimide:

A solution of copper (II) chloride (134 μg, 1.0 μmol) and triethylamine (151 μg, 1.5 μmol) in acetonitrile (40 μL) was added to the N-propagyl maleimide (135 μg, 1.0 μmol). After 5 min the resulting solution was added to a mixture of dansyl ethyl azide (319 μg, 1.0 μmol) in acetonitrile (20 μL) and [¹²⁵I]NaI (20-40 MBq) in water (6.0 μL). The solution was heated at 60° C. for 90 min. The reaction was cooled to room temperature and diluted with water and acetonitrile (1:1, 1.0 mL) and the resulting solution was purified by HPLC using a ZORBAX column (300SB-C18, 9.4×250 mm, 5 μm) with the following eluent: water (0.1% formic acid) as solvent A and methanol (0.1% formic acid) as solvent B, going from 40% of B to 90% of B in 8 min and going back to 40% of B in 2 min and remaining at 40% of B for an additional 2 min with a flow rate of 3 mL/min. The retention time of the title compound was 9.09 min. The labelled compound co-eluted with the non-radioactive reference compound.

1. A process for producing a triazole derivative represented by the formula (I) 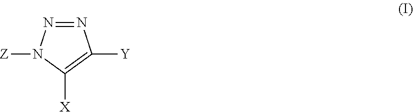
wherein
X represents a radioisotopic halogen atom; and
Y and Z each represent, independently of one another, a moiety which is
a) a label, a chelating agent, a biologically active moiety or a moiety enabling purification of the derivative; or
b) a reactive linker for linking the triazole derivative represented by the formula (I) to a label, a chelating agent, a biologically active moiety or a moiety enabling purification of the derivative;
which process comprises (B) reacting an alkyne represented by the formula (II′) 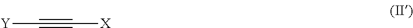
with an azide represented by the formula (III)
Z—N₃ (III)
said process being carried out in the presence of Cu(II) ions and a base.

2. The process according to claim 1, which process further comprises (A) reacting a terminal alkyne represented by the formula (II) 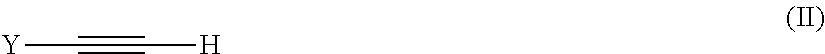
with a radioisotopic halogen anion, in the presence of Cu(II) ions and a base, wherein
Y is as defined in claim 1, to obtain thereby an alkyne represented by the formula (II′) as defined in claim 1.

3. The process according to claim 2, wherein (A) and (B) are carried out in a single synthetic procedure that is carried out without isolating the alkyne represented by the formula (II′) from the reaction medium.

4. The process according to claim 1, wherein the radioisotopic halogen atom is selected from ¹²³I, ¹²⁴I, ¹²⁵I and ¹³¹I.

5. The process according to claim 1, wherein each biologically active moiety is selected independently from a moiety capable of binding to a biomolecule of interest in vivo, a moiety capable of modifying the physicochemical properties of the derivative in vivo, a peptide, a protein, an antibody or antibody fragment, a DNA or DNA analogue, a chemical entity that is sensitive to changes in in vivo environment and a drug moiety.

6. The process according to claim 2, wherein at least one of (A) and (B) is carried out in the presence of a Cu(II) salt selected from CuCl₂, CuBr₂, CuSO₄, a Cu(II) carboxylic acid salt and Cu(II) oxalate.

7. The process according to claim 2, wherein at least one of (A) and (B) is carried out in the presence of an oxidising agent capable of oxidising Cu(I) ions to Cu(II) ions in situ.

8. The process according to claim 7, wherein the oxidising agent capable of oxidising Cu(I) ions to Cu(II) ions in situ is selected from N-iodosuccinimide, N-bromosuccinimide, quinine, H₂O₂ and FeCl₃.

9. The process according to claim 1, wherein the base is triethylamine.

10. The process according to claim 1, wherein the base is used in an amount of at least one mole per one mole of Cu(II) ions.

11. The process according to claim 1, wherein the base is used in an amount of at most two moles per one mole of Cu(II) ions.

12. The process according to claim 2 wherein at least one of (A) and (B) is carried out in the presence of a ligand capable of co-ordinating to Cu(II) ions.

13. The process according to claim 1 wherein at least one of Y and Z represents a reactive linker and said process further comprises linking, independently, each said reactive linker to a label, a chelating agent, a biologically active moiety, or a moiety enabling purification of the derivative.

14. The process according to claim 1, wherein Y and Z each represent, independently of one another, a moiety which is a label, a chelating agent, a biologically active moiety or a moiety enabling purification of the derivative.

15. (canceled)

16. A triazole derivative represented by the formula (I) as defined in claim 1, and wherein Y and Z each represent, independently of one another, a moiety which is a label, a chelating agent, a biologically active moiety or a moiety enabling purification of the derivative.

17. A diagnostic method practised on a human or animal patient, which comprises administering a triazole derivative as defined in claim 16 to the patient.

18. The method according to claim 17, wherein said diagnostic method comprises a method of medical imaging.

19. The method according to claim 18, wherein said method of medical imaging is selected from imaging with PET, SPECT, MRI, CT, ultrasound and optical techniques.

20. A method of imaging carried out on a human or animal subject, which method comprises measuring the distribution in vivo of a triazole derivative as defined in claim 16.

21. The method according to claim 20, which further comprises a step of administering said triazole derivative to the human or animal subject prior to said step of measuring the distribution in vivo of said triazole derivative.

22. An in vitro assay method which comprises measuring the distribution in a biological sample of a triazole derivative as defined in claim 16.

23. A method of treatment of a human or animal patient, which comprises administering a triazole derivative as defined in claim 16 to the patient and wherein said triazole derivative comprises at least one drug moiety.

24. The method according to claim 23, wherein said method of treatment comprises radiotherapy.

25. A process for producing an alkyne represented by the formula (II′) as defined in claim 1, which process comprises reacting a terminal alkyne represented by the formula (II) as defined in claim 2 with a radioisotopic halogen anion, said process being carried out in the presence of Cu(II) ions and a base.

26. The process according to claim 1, which is carried out in the presence of a Cu(II) salt selected from CuCl₂, CuBr₂, CuSO₄, a Cu(II) carboxylic acid salt and Cu(II) oxalate.

27. The process according to claim 1, which is carried out in the presence of an oxidising agent capable of oxidising Cu(I) ions to Cu(II) ions in situ.

28. The process according to claim 27, wherein the oxidising agent capable of oxidising Cu(I) ions to Cu(II) ions in situ is selected from N-iodosuccinimide, N-bromosuccinimide, quinine, H₂O₂ and FeCl₃.

29. The process according to claim 1, which is carried out in the presence of a ligand capable of co-ordinating to Cu(II) ions.
